(19) United States
(12) Patent Application Publication
(10) Pub. No.: US 2024/0342190 A1
(43) Pub. Date: Oct. 17, 2024

(54) METHODS FOR REDUCING ISCHEMIA-REPERFUSION INJURIES

(71) Applicants: BioMimetix JV, LLC, Englewood, CO (US); Duke University, Durham, NC (US)

(72) Inventors: James D. Crapo, Englewood, CO (US); David Warner, Chapel Hill, NC (US); Huaxin Sheng, Chapel Hill, NC (US); Ines Batinic-Haberle, Durham, NC (US); Ivan Spasojevic, Durham, NC (US)

(21) Appl. No.: 18/633,760

(22) Filed: Apr. 12, 2024

Related U.S. Application Data
(60) Provisional application No. 63/495,891, filed on Apr. 13, 2023.

(51) Int. Cl. A61K 31/555; A01N 1/02; A61K 9/00; A61P 9/10

(52) U.S. Cl. CPC A61K 31/555; A01N 1/0226; A61K 9/0019; A61P 9/10

(57) ABSTRACT

Described herein is a method for reducing an ischemia-reperfusion injury in a subject, the method comprising intra-arterially administering a redox active compound that comprises a metal to the subject. Also described herein is a method for increasing the time of viability of an organ awaiting transplantation and/or reducing transplant failure in a subject, the method comprising contacting the organ and a redox active compound that comprises a metal.

### RELATED APPLICATION INFORMATION

This application claims the benefit of U.S. Provisional Patent Application Ser. No. 63/495,891, filed Apr. 13, 2023, the disclosure of which is incorporated herein by reference in its entirety.

### STATEMENT OF GOVERNMENT SUPPORT

This invention was made with government support under Grant No. R41NS110252-01A1 awarded by the National Institutes of Health and Grant No. P30CA014236 awarded by the National Cancer Institute. The government has certain rights in the invention.

### FIELD OF THE INVENTION

The present invention is directed to methods for reducing ischemia-reperfusion injuries in a subject and to compounds and compositions for intra-arterial administration to a subject. The present invention is also directed to methods of increasing viability of an organ awaiting transplant and/or reducing transplant failure in a subject.

### BACKGROUND

Reperfusion to ischemic tissues poses an interesting medical challenge for physicians. Early restoration of blood flow is critical for the treatment of an ischemic stroke or heart attack. For example, a blood clot that blocks a cerebral artery can immediately lead to neurologic and functional deficits as any brain tissue within this arterial territory is metabolically compromised. Thus, thrombectomies and embolectomies are often performed as soon as possible after diagnosis in order to remove the offending clot and restore blood flow. However, rapid reperfusion of ischemic tissues can lead to local inflammation and increases in reactive-oxidative species (ROS), which ultimately results in significant cell death. In addition, organ transplants often suffer from similar ischemia-reperfusion injuries after blood flow is restored to the donor organ. This can lead to challenging acute rejections. Thus, there is an urgent need to have new methods developed to overcome the obstacles that arise from such ischemia-reperfusion injuries.

### SUMMARY

One aspect of the present invention is directed to a method of reducing an ischemia-reperfusion injury in a subject in need thereof, the method comprising: intra-arterially administering a redox active compound that comprises a metal to the subject, thereby reducing the ischemia-reperfusion injury in the subject. In some embodiments, the method improves the ischemia-reperfusion injury outcome in the subject, wherein the outcome comprises a reduced number and/or severity of one or more neurological deficit(s), a reduced amount of ischemic tissue, and/or a reduced infarct volume. In some embodiments, the ischemia-reperfusion injury is caused by a stroke, a heart attack, and/or an organ transplantation.

Another aspect of the present invention is directed to a method of increasing viability of an organ awaiting transplantation and/or reducing transplant failure in a subject, the method comprising contacting the organ and a redox active compound that comprises a metal.

It is noted that aspects described with respect to one embodiment may be incorporated in different embodiments although not specifically described relative thereto.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1, Panel A is a graph showing perioperative blood pressure that was measured during surgery and that the BMX-001 group had a lower blood pressure at 5 min vs 45 min post-treatment. FIG. 1, Panel B is a graph showing that ischemic injury induced a significant body weight loss at 24 hours post-stroke and that BMX-001 treatment promoted post-stroke body weight recovery (p=0.02 in BMX-001 and p=0.27 in vehicle; body weight at 7 Days vs body weight at 24 hours). FIG. 1, Panel C is a graph showing that neurologic scores were significantly improved by BMX-001 treatment. FIG. 1, Panel D is a graph showing that infarct volume was significantly improved by BMX-001 treatment. Neurologic scores were expressed as median±IQR, and other data were expressed as mean±SD, *p<0.05, **p<0.01.

FIG. 2, Panel A is a graph showing that neurologic scores were evaluated at day 7 and 28 post-stroke and that theBMX-001-treated group had a better neurologic score at day 7 (p=0.04) and day 28 (p=0.04) compared to the vehicle group FIG. 2, Panel B is a graph showing that infarct volumes were measured at day 28 and were smaller in BMX-001-treated group and no intragroup difference was detected. Neurologic scores were expressed as median±IQR, and infarct volume was expressed as mean±SD, *p<0.05.

FIG. 3, Panel A is a graph showing that the vehicle group had somewhat slower body weight recovery. FIG. 3, Panel B is a graph showing that neurological deficits were significantly improved by BMX-001 treatment on both day 7 and day 28 post-stroke. FIG. 3, Panel C is a graph showing that no intra-group difference was found in infarct volume although the vehicle group had a larger infarct. Neurologic scores were expressed as median±IQR, and other data were expressed as mean±SD, **p<0.01.

FIG. 4, Panel A is a graph showing that the vehicle group had a somewhat slower body weight recovery. FIG. 4, Panel B is a graph showing that neurological deficits were significantly improved by BMX-001 treatment at day 28 post-stroke. FIG. 4, Panel C is a graph showing that no intra-group difference was found in infarct volume although the vehicle group had a larger infarct. Neurologic scores were expressed as median±IQR, and other data were expressed as mean±SD, *p<0.05.

FIG. 5, Panel A is a graph showing that body weight was monitored daily for the first 7 days after stroke. FIG. 5, Panel B is a graph showing that neurologic functions were evaluated on day 7 and 28 post-stroke and that BMX-001 treatment significantly attenuated neurologic deficits. FIG. 5, Panel C is a graph showing that infarct volume was measured 28 days after stroke and that BMX-001 treatment significantly reduced infarct volume. Neurologic scores were expressed as median±IQR, and other data were expressed as mean±SD, *p<0.05, **p<0.01.

FIG. 6, Panel A is a graph showing that Evan blue content was measured in the brain after intracarotid infusion of vehicle, BMX-001 and Mannitol and that BMX-001 did not affect the BBB while Mannitol did increase the blood brain barrier (BBB) permeability. FIG. 6, Panel B is a graph showing that another set of rats was given vehicle, BMX-001 and Solu-Medrol, and that body weight was monitored day 1, 2 and 3 after intracarotid infusion. FIG. 6, Panel C is a series of microscope images showing that Solu-Medrol caused sickness in rats and induced brain damage, while no histologic damage was found in BMX-001 and vehicle group. Data were expressed as mean±SD, **p<0.01.

### DETAILED DESCRIPTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention.

All publications, patent applications, patents and other references mentioned herein are incorporated by reference in their entirety. In case of a conflict in terminology, the present specification is controlling.

As used in the description of the invention and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

As used herein, the terms “increase,” “increasing,” “enhance,” “enhancing,” “improve” and “improving” (and grammatical variations thereof) describe an elevation of about 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of about 1%, 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

Like numbers refer to like elements throughout. In the figures, the thickness of certain lines, layers, components, elements or features may be exaggerated for clarity. The abbreviations “FIG. and “FIG.” for the word “Figure” can be used interchangeably in the text and figures.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, in “direct contact” with, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

“Alkyl” as used herein alone or as part of another group, refers to a straight or branched chain saturated hydrocarbon containing from 1 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The term “akyl” or “lower alkyl” is intended to include both substituted and unsubstituted alkyl or lower alkyl unless otherwise indicated and these groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Alkenyl” as used herein alone or as part of another group, refers to a straight or branched chain hydrocarbon containing from 1 to 20 carbon atoms (or in loweralkenyl 1 to 4 carbon atoms) which include 1 to 19 double bonds in the chain. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “loweralkenyl” is intended to include both substituted and unsubstituted alkenyl or loweralkenyl unless otherwise indicated and these groups may be substituted with groups as described in connection with alkyl and loweralkyl above.

“Alkynyl” as used herein alone or as part of another group, refers to a straight or branched chain hydrocarbon containing from 1 to 20 carbon atoms (or in loweralkynyl 1 to 4 carbon atoms) which include 1 triple bond in the normal chain. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralknynyl unless otherwise indicated and these groups may be substituted with the same groups as set forth in connection with alkyl and loweralkyl above.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. In some embodiments, cycloalkyl refers to only a saturated cyclic hydrocarbon group containing from 3, 4 or 5 to 6, 7 or 8 carbons (which carbons may be replaced in a heterocyclic group as discussed below). The term “cycloalkyl” is generic and intended to include heterocyclic groups as discussed below unless specified otherwise.

“Heterocyclic group” or “heterocyclo” as used herein alone or as part of another group, refers to an aliphatic (e.g., fully or partially saturated heterocyclo) or aromatic (e.g., heteroaryl) monocyclic-or a bicyclic-ring system having as ring members atoms of at least two different elements. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and lower alkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroaryl” as used herein is as described in connection with heterocyclo above.

“Alkoxy” as used herein alone or as part of another group, refers to an alkyl or loweralkyl group, as defined herein (and thus including substituted versions such as polyalkoxy), appended to the parent molecular moiety or another group through an oxy group, —O—. Representative examples of alkoxy include, but are not limited to, methoxy, ethoxy, propoxy, 2-propoxy, butoxy, tert-butoxy, pentyloxy, hexyloxy and the like. “Alkoxyalkyl” as used herein alone or as part of another group, refers to an alkyl or loweralkyl group, as defined herein (and thus including substituted versions such as polyalkoxy), appended to another alkyl or loweralkyl group through an oxy group, —O—, such as, but not limited to, —CH₂CH₂OCH₂CH₂CH₂CH₃.

“H” refers to a hydrogen atom. “C” refers to a carbon atom. “N” refers to a nitrogen atom. “O” refers to an oxygen atom. “Br” refers to a bromine atom. “Cl” refers to a chlorine atom. “I” refers to an iodine atom. “F” refers to a fluorine atom. “Mn” refers to a manganese atom. “Fe” refers to an iron atom. “Cu” refers to a copper atom. “Co” refers to a cobalt atom. “Ni” refers to a nickel atom. “Zn” refers to a zinc atom.

“Halo” as used herein refers to any suitable halogen, including —F, —Cl, —Br, and —I.

“Mercapto” as used herein refers to an —SH group.

“Azido” as used herein refers to an —N3 group.

“Cyano” as used herein refers to a —CN group.

“Formyl” as used herein refers to a —C(O)H group.

“Carboxylic acid” as used herein refers to a —C(O)OH group.

“Hydroxyl” as used herein refers to an —OH group.

“Nitro” as used herein refers to an —NO₂ group.

“Acyl” as used herein alone or as part of another group refers to a —C(O)R radical, where R is any suitable substituent such as aryl, alkyl, alkenyl, alkynyl, cycloalkyl or other suitable substituent as described herein.

“Alkylthio” as used herein alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Amino” as used herein means the radical —NH₂.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and Rb is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NRₐR_{b} radical, where Ra and Rb are any suitable substituent such as alkyl, cycloalkyl, alkenyl, alkynyl or aryl.

“Sulfoxyl” as used herein refers to a compound of the formula —S(O)R, where R is any suitable substituent such as alkyl, cycloalkyl, alkenyl, alkynyl or aryl.

“Sulfonyl” as used herein refers to a compound of the formula —S(O)(O)R, where R is any suitable substituent such as alkyl, cycloalkyl, alkenyl, alkynyl or aryl.

“Sulfonate” as used herein refers to a compound of the formula —S(O)(O)OR, where R is any suitable substituent such as alkyl, cycloalkyl, alkenyl, alkynyl or aryl.

“Sulfonic acid” as used herein refers to a compound of the formula —S(O)(O)OH.

“Sulfonamide” as used herein alone or as part of another group refers to a —S(O)₂NRₐR_{b} radical, where Ra and Rb are any suitable substituent such as H, alkyl, cycloalkyl, alkenyl, alkynyl or aryl.

“Urea” as used herein alone or as part of another group refers to an —N(R_{c})C(O)NRₐR_{b} radical, where Rₐ, R_{b} and R_{c} are any suitable substituent such as H, alkyl, cycloalkyl, alkenyl, alkynyl or aryl.

“Alkoxyacylamino” as used herein alone or as part of another group refers to an —N(Rₐ)C(O)OR_{b} radical, where Rₐ, R_{b} are any suitable substituent such as H, alkyl, cycloalkyl, alkenyl, alkynyl or aryl.

“Aminoacyloxy” as used herein alone or as part of another group refers to an —OC(O)NRₐR_{b} radical, where Rₐ and R_{b} are any suitable substituent such as H, alkyl, cycloalkyl, alkenyl, alkynyl or aryl.

Unless indicated otherwise, nomenclature used to describe chemical groups or moieties as used herein follow the convention where, reading the name from left to right, the point of attachment to the rest of the molecule is at the right-hand side of the name. For example, the group “(C₁₋₃ alkoxy) C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, where a mono or bivalent group is described by its chemical formula, including one or two terminal bond moieties indicated by “−,” it will be understood that the attachment is read from left to right.

Unless otherwise stated, structures depicted herein are meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

“Pharmaceutically acceptable” as used herein means that the compound, anion, or composition is suitable for administration to a subject to achieve the treatments described herein, without unduly deleterious side effects in light of the severity of the disease and necessity of the treatment.

Provided according to embodiments of the present invention are methods of reducing an ischemia-reperfusion injury in a subject. A method of the present invention may comprise intra-arterially administering to a subject a redox active compound that comprises a metal. In some embodiments, the redox active compound that comprises a metal is a meso-substituted metalloporphyrin. One or more (e.g., 1, 2, 3, 4, or more) different redox active compound(s) may be administered to a subject in a method of the present invention and/or may be present in a composition of the present invention.

Exemplary redox active compounds of the present invention include, but are not limited to, those having a structure of Formula I:

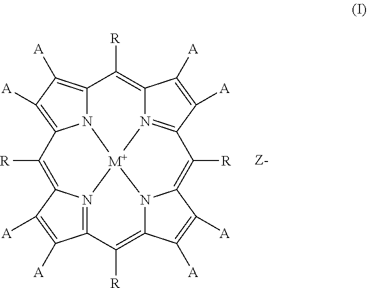

wherein:
each R is, independently, a substituted or unsubstituted aryl, heteroaryl, cycloalkyl, or heterocycloalkyl, optionally wherein the aryl, heteroaryl, cycloalkyl, or heterocycloalkyl is substituted and optionally includes at least one fluorine (e.g., 1, 2, 3, 4, 5, 6, or more fluorine atoms);
each A is an independently selected hydrogen or an electron-withdrawing or electron donating group (e.g., a halogen, —NO₂ or —CHO);
M is a metal (e.g., manganese, iron, copper, cobalt, nickel or zinc); and
Z⁻ is a counterion (e.g., an anion such as a halogen ion (e.g., a chloride, fluoride, bromide, and/or iodide ion) and/or a conjugate base of an acid (e.g., an oleate)).

In some embodiments, each R in the compound of Formula I is a heteroaryl or heterocycloalkyl. In some embodiments, each R in the compound of Formula I is a heteroaryl or heterocycloalkyl that each independently include at least one or two nitrogen atoms in the heterocyclic ring, optionally wherein each R is independently selected from a pyrrolyl, imidazolyl, triazolyl, pyridyl, pyrimidyl, triazinyl, oxazolyl, thiazolyl, oxazinyl, thiazinyl, and/or oxathiazinyl, each of which may be substituted or unsubstituted. In some embodiments, in a compound of Formula I, each R is a heteroaryl or heterocycloalkyl that includes at least one nitrogen atom (or in some embodiments at least two nitrogen atoms) that is substituted (e.g., quaternized) with a substituent such as described in connection with heterocyclic groups above (e.g., substituted with alkyl, alkoxyalkyl, etc.). In some embodiments, Z⁻ in a compound of Formula I is a halogen such as chlorine. In some embodiments, Z⁻ in a compound of Formula I is a conjugate base of an acid such as an oleate. In some embodiments, each R in the compound of Formula I is a heteroaryl or heterocycloalkyl that are each independently substituted with an alkoxy. In some embodiments, each R in the compound of Formula I is a heteroaryl or heterocycloalkyl that are each independently substituted with an alkoxy that includes one or more fluorine atoms.

In some embodiments, a redox active compound of the present invention is an alkyl substituted imidazole porphyrin. Exemplary alkyl substituted imidazole porphyrins include, but are not limited to, those having a structure of Formula A1 or A2:

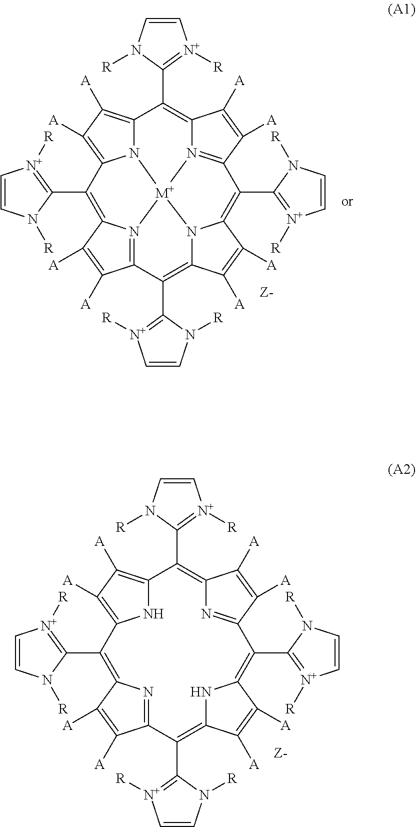

wherein:
each R is, independently, a C1-12 alkyl (straight chain or branched), optionally wherein the C1-12 alkyl is substituted with at least 1 fluorine (e.g., 1, 2, 3, 4, 5, 6, or more fluorine atoms);
each A is, independently, a hydrogen or an electron withdrawing group (e.g., a halogen, —NO₂ or —CHO);
M is a metal selected from the group consisting of manganese, iron, copper, cobalt, nickel and zinc; and
Z⁻ is a counterion.

In some embodiments, a redox active compound of the present invention is an alkyl substituted pyridine porphyrin. Exemplary alkyl substituted pyridine porphyrins include, but are not limited to, those having a structure of Formula B1:

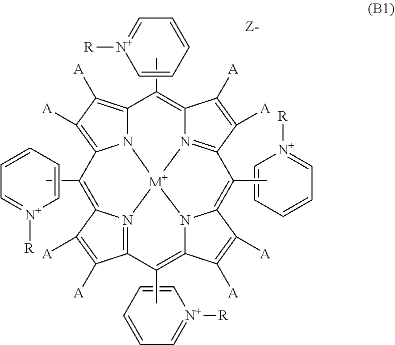

wherein:
each R is, independently, a C1-12 alkyl or C1-C12 alkoxyalkyl, optionally wherein the C1-12 alkyl or C1-C12 alkoxyalkyl is substituted with at least 1 fluorine (e.g., 1, 2, 3, 4, 5, 6, or more fluorine atoms);
each A is, independently, a hydrogen or an electron withdrawing group;
M is a metal selected from the group consisting of manganese, iron, copper, cobalt, and nickel; and
Z⁻ is a counterion;
In some embodiments, a redox active compound of the present invention is MnTE-2-PyP⁵⁺ and has the structure:

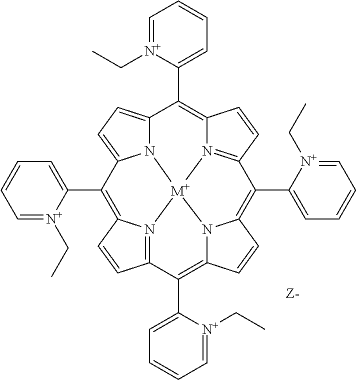

wherein Z⁻ is a counterion (e.g., a halogen ion) and M⁺ is manganese.

In some embodiments, a redox active compound of the present invention is an alkyl substituted pyridine porphyrin. Exemplary alkyl substituted pyridine porphyrins include, but are not limited to, those having a structure of Formula C1:

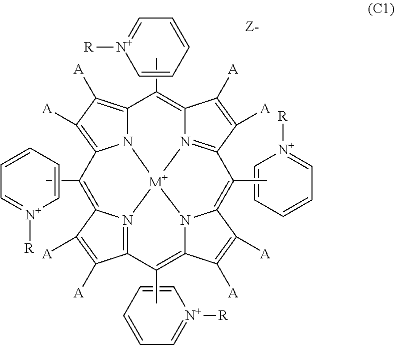

wherein:
each R is, independently, a hydrogen or —(CH₂)ₘCH₂OX, optionally wherein the —(CH₂)ₘCH₂OX is substituted with at least 1 fluorine (e.g., 1, 2, 3, 4, 5, 6, or more fluorine atoms);
m is 1 or 2;
X is a C1-12 alkyl;
each A is, independently, a hydrogen or an electron withdrawing group;
M is a metal selected from the group consisting of manganese, iron, copper, cobalt, and nickel, and
Z⁻ is a counterion.

In some embodiments, a redox active compound of the present invention has the structure:

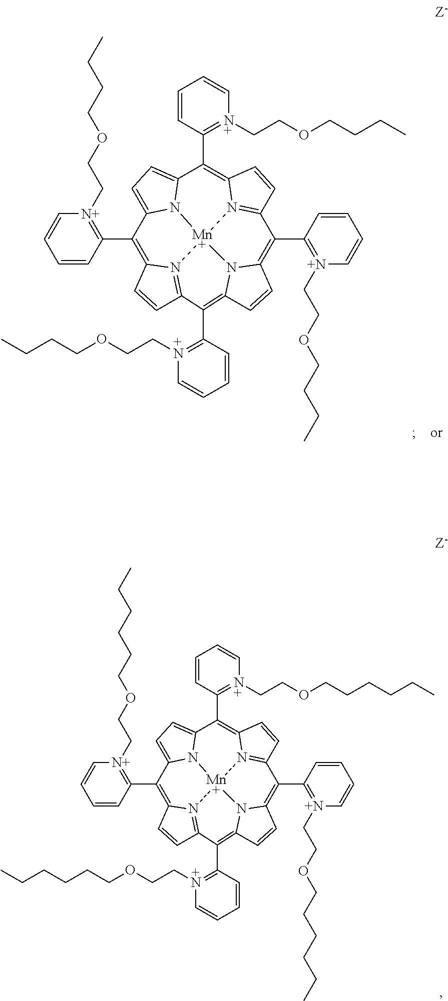

wherein Z⁻ is a counterion.

In some embodiments, a redox active compound of the present invention is BMX-001 and has the structure:

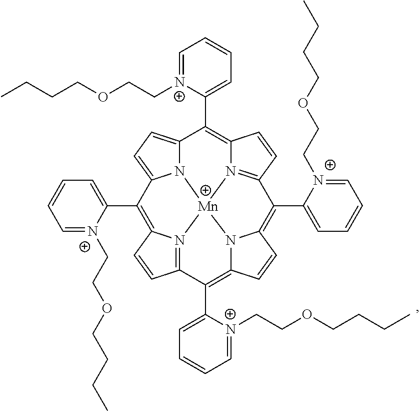

wherein Z⁻ is a counterion.

A redox active compound of the present invention may be prepared in the form of a salt such as a pharmaceutically acceptable salt, e.g., to provide a compound or composition including a counterion as noted above. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and/or (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

A redox active compound of the present invention may be formulated for administration in a pharmaceutical carrier in accordance with known techniques. See, e.g., Remington, The Science And Practice of Pharmacy (9th Ed. 1995). In the manufacture of a pharmaceutical formulation according to the invention, a redox active compound of the present invention (including the physiologically acceptable salts thereof) may be admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a solution, which may contain from about 0.01%, 0.1%, 0.5%, 1%, 5%, or 10% to about 20%, 30%, 40%, 50%, 60%, 70%, 80%, 90% 95% or 99% by weight of the redox active compound. In some embodiments, the redox active compound is present in a composition (e.g., a solution such as an aqueous solution) in amount of about 0.01% or 0.1% to about 0.25% 0.5%, 1%, 5%, or 10% by weight. One or more redox active compound(s) may be included in a formulation of the present invention, which may be prepared by any of the well-known techniques of pharmacy comprising admixing the components, optionally including one or more accessory ingredients. In some embodiments, a pharmaceutical carrier used in the present invention may be water, saline, and/or a buffer.

In addition to active agent(s), a pharmaceutical composition of the present invention may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, a composition of the present invention may contain a microbial preservative. Useful microbial preservatives include, but are not limited to, methylparaben, propylparaben, and benzyl alcohol. A microbial preservative is typically employed when the formulation is placed in a vial designed for multi-dose use. Of course, as indicated, a pharmaceutical composition of the present invention may be lyophilized using techniques well known in the art.

The formulations of the invention include those suitable for oral, rectal, topical, buccal (e.g., sub-lingual), vaginal, parenteral (e.g., subcutaneous, intramuscular, intradermal, intra-arterial, or intravenous), topical (i.e., both skin and mucosal surfaces, including airway surfaces) and transdermal administration, although the most suitable route in any given case will depend on the nature and severity of the condition being treated and on the nature of the particular redox active compound which is being used.

Formulations suitable for oral administration may be presented in discrete units, such as capsules, cachets, lozenges, or tablets, each containing a predetermined amount of a redox active compound of the present invention; as a powder or granules; as a solution or a suspension in an aqueous or non-aqueous liquid; or as an oil-in-water or water-in-oil emulsion. Such formulations may be prepared by any suitable method of pharmacy which includes the step of bringing into association a redox active compound of the present invention and a suitable carrier (which may contain one or more accessory ingredients as noted above). In general, the formulations of the invention are prepared by uniformly and intimately admixing a redox active compound of the present invention with a liquid or finely divided solid carrier, or both, and then, if necessary, shaping the resulting mixture. For example, a tablet may be prepared by compressing or molding a powder or granules containing a redox active compound of the present invention, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising a redox active compound of the present invention in a flavored base, usually sucrose and acacia or tragacanth; and pastilles comprising a redox active compound of the present invention in an inert base such as gelatin and glycerin or sucrose and acacia.

Formulations suitable for rectal administration are preferably presented as unit dose suppositories. These may be prepared by admixing a redox active compound of the present invention with one or more conventional solid carriers, for example, cocoa butter, and then shaping the resulting mixture.

Formulations of the present invention include those that are suitable for parenteral administration (e.g., subcutaneous, intramuscular, intradermal, intra-arterial, or intravenous administration) and comprise sterile aqueous and non-aqueous injection solutions of a redox active compound of the present invention, which preparations are preferably isotonic with the blood of the intended recipient. These preparations may contain anti-oxidants, buffers, bacteriostats and/or solutes which render the formulation isotonic with the blood of the intended recipient. Aqueous and non-aqueous sterile suspensions may include suspending agents and/or thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a redox active compound of the present invention, or a salt thereof, in a unit dosage form in a sealed container. The redox active compound or salt may be provided in the form of a lyophilizate which is capable of being reconstituted with a suitable pharmaceutically acceptable carrier to form a liquid composition suitable for injection thereof into a subject. In some embodiments, the pharmaceutically acceptable carrier is saline. The unit dosage form may comprise from about 3 μg to about 10 grams of the redox active compound or salt. When the redox active compound or salt is substantially water-insoluble, a sufficient amount of emulsifying agent that is physiologically acceptable may be employed in sufficient quantity to emulsify the redox active compound or salt in an aqueous carrier. One such useful emulsifying agent is phosphatidyl choline.

In some embodiments, a method of the present invention comprises administering a therapeutically effective amount of a redox active compound of the present invention to a subject. As used herein, the term “therapeutically effective amount” refers to an amount of a redox active compound of the present invention that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with an ischemia-reperfusion injury and/or transplant failure is achieved and/or there is a delay in the progression of the symptom. In some embodiments, the severity of a symptom associated with an ischemia-reperfusion injury may be reduced in a subject compared to the severity of the symptom in the absence of a method of the present invention.

In some embodiments, a redox active compound of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with an ischemia-reperfusion injury and/or a reduction in the severity of the onset of symptom associated with an ischemia-reperfusion injury and/or transplant failure relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention.

In some embodiments, a redox active compound of the present invention may be administered in a prevention effective amount. A “prevention effective” amount as used herein is an amount that is sufficient to prevent (as defined herein) a symptom associated with an ischemia-reperfusion injury and/or transplant failure in a subject. Those skilled in the art will appreciate that the level of prevention need not be complete, as long as some benefit is provided to the subject. In some embodiments, a prevention effective amount may be achieved by administering a composition of the present invention.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g., rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention. For example, the subject is suffering from an ischemia-reperfusion injury, has had an ischemia-reperfusion injury, or has findings typically associated with having an ischemia-reperfusion injury. In some embodiments, the subject is awaiting an organ transplant or has received an organ transplant.

In some embodiments, a redox active compound of the present invention is micronized prior to inclusion in a composition of the present invention, optionally by jet milling, ball milling, spray drying, controlled crystallization, and/or the like. In some embodiments, a redox active compound of the present invention, prior to inclusion in a composition of the present invention and/or in a composition of the present invention, has a D90 particle size of less than 50 microns, optionally a D90 of about 45, 40, or 30 microns. In some embodiments, a redox active compound of the present invention, prior to inclusion in a composition of the present invention and/or in a composition of the present invention, has a D50 particle size of about 0.5 microns to about 10 microns. In some embodiments, a redox active compound of the present invention, prior to inclusion in a composition of the present invention and/or in a composition of the present invention, has a D10 particle size of about 3, 3.5, or 4 microns to about 4.5, 5, 5.5, or 6 microns. In some embodiments, a redox active compound of the present invention, prior to inclusion in a composition of the present invention and/or in a composition of the present invention, has a D90 particle size of less than 3 microns, optionally less than 2 microns. In some embodiments, a redox active compound of the present invention, prior to inclusion in a composition of the present invention and/or in a composition of the present invention, has a D50 of about 0.5 or 0.75 microns to about 1, 1.5, 2, 2.5 or 3 micron(s). In some embodiments, a redox active compound of the present invention, prior to inclusion in a composition of the present invention and/or in a composition of the present invention, has a D10 particle size of about 0.1, 0.2, or 0.3 microns to about 0.4, 0.5, or 0.6 microns. In some embodiments, a redox active compound of the present invention, prior to inclusion in a composition of the present invention and/or in a composition of the present invention, has a D10 of about 0.1, 0.2, or 0.3 microns to about 0.4, 0.5, or 0.6 microns, a D50 of about 0.5, 0.75, 1, 1.5, 2, 2.5 or 3 micron(s), and/or a D90 of about 1 or 1.5 micron(s) to about 2 or 2.5 microns. In some embodiments, a redox active compound of the present invention, prior to inclusion in a composition of the present invention and/or in a composition of the present invention, has a median particle size of about 1, 2, 3, or 4 micron(s) to about 5, 6, 7, 8, 9, 10, 11, 12 13, 14, or 15 microns. In some embodiments, a redox active compound of the present invention, prior to inclusion in a composition of the present invention and/or in a composition of the present invention, has a median particle size of less than about 5 microns.

A method of the present invention may comprise administering and/or contacting a redox active compound of the present invention to ischemic tissue. In some embodiments, the ischemic tissue may be in the brain and/or heart of the subject and/or the redox active compound may be administered (e.g., directly administered and/or contacted) to the brain and/or heart. In some embodiments, a redox active compound of the present invention may be administered such that it is delivered directly to a clot, such as a blood clot, in the subject. In some embodiments, administering the redox active compound comprises administering the redox active compound directly to a site of an ischemia-reperfusion injury (e.g., directed to the site of a clot in the subject). In some embodiments, administering the redox active compound comprises administering the redox active compound directly to a site of a stroke and/or heart attack in a subject.

In some embodiments, a redox active compound of the present invention may be intra-arterially administered using an intra-arterial catheter. In some embodiments, the intra-arterial administration may be administration into the carotid artery of a subject, such as infusing and/or injecting a redox active compound of the present invention into the carotid artery of the subject. In some embodiments, the intra-arterial administration may comprise administering the redox active compound into the middle cerebral artery of a subject. In some embodiments, a redox active compound of the present invention may be intra-arterially administered to cross the blood brain barrier of a subject.

In some embodiments, intra-arterial administration of a redox active compound of the present invention may be performed after removal of a catheter and/or filament and/or after reperfusion in a subject. In some embodiments, intra-arterial administration of a redox active compound of the present invention may be performed prior to the removal of a catheter and/or filament and/or prior to reperfusion in a subject.

In some embodiments, the intra-arterially administering of a method of the present invention is performed prior to, during, and/or after a thrombectomy, embolectomy, angioplasty, and/or cardiac catheterization that is performed on the subject and/or is performed prior to, during, and/or after administration of tissue plasminogen activator (t-PA) to the subject. In some embodiments, the intra-arterially administering is performed concurrently with a thrombectomy, embolectomy, angioplasty, and/or cardiac catheterization that is performed on the subject.

In some embodiments, a redox active compound of the present invention may be intra-arterially administered to a subject in an amount of about 1, 3, 5, 10, 25, 50, 75, or 100 μg/kg to about 150, 200, 250, 300, 350, 400, 450, or 500 μg/kg. For example, the redox active compound may be intra-arterially administered to a subject in an amount of about 1, 3, 5, 10, 15, 20, 25, 30, 35, 40, 45, 50, 55, 60, 65, 70, 75, 80, 85, 90, 95, 100, 150, 200, 250, 300, 350, 400, 450, or 500 μg/kg. In some embodiments, the redox active compound may be intra-arterially administered to a subject in an amount of about 5, 10, 15, 20, 25, 30, 35, 40, 45, or 50 μg/kg to about 55, 60, 65, 70, 75, 80, 85, 90, 95, 100 μg/kg. In some embodiments, the amount of the redox active compound that is intra-arterially administered to the subject is adjusted based on the location of the administration in the subject, the blood vessel through which the redox active compound is administered, and/or the ischemia-reperfusion injury in the subject.

In some embodiments, a redox active compound of the present invention may be administered subcutaneously and/or intravenously to a subject. A composition comprising the redox active compound may include the redox active compound in a concentration of about 0.1, 0.5, 1, 2, or 5 mg/mL to about 10, 15, 20, or 25 mg/mL.

A method of the present invention may comprise administering a loading dose of a redox active compound of the present invention to a subject. In some embodiments, the loading dose is an initial dose and/or is administered one time. In some embodiments, the loading dose may be administered to the subject in an amount of about 5 mg to about 50 mg. For example, the subject may be administered about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg of the redox active compound as loading dose. In some embodiments, the loading dose may be administered to the subject within about 1, 5, 10, 30, 45, or 60 minute(s) to about 75, 90, 160, 180, 240, 300, or 360 minutes or 7, 8, 9, 10, 11, or 12 hours of the ischemia-reperfusion injury. In some embodiments, the loading dose may be subcutaneously and/or intravenously administered to the subject. In some embodiments, a loading dose and t-PA may be administered to a subject within about 1 hour of each other. In some embodiments, a loading dose is administered to a subject such that the subject's daily dosage of the redox active compound is in an amount of about 5 mg to about 50 mg of the redox active compound, optionally about 28 mg of the redox active compound, in a 24-hour period, optionally wherein the loading dose is a single, daily dose.

A method of the present invention may comprise administering a maintenance dose of a redox active compound of the present invention to a subject. In some embodiments, the maintenance dose may be administered to the subject in an amount of about 5 to about 50 mg of the redox active compound per week. For example, in administering the maintenance dose, the subject may be administered about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg of the redox active compound per week. In some embodiments, a maintenance dose is administered to a subject such that the subject's average daily dosage of the redox active compound is in an amount of about 1, 2, 3, or 4 mg to about 5, 6, 7, 8, 9, or 10 mg of the redox active compound, optionally about 4 mg of the redox active compound. In some embodiments, the maintenance dose is administered to the subject in an amount that is about 25%, 30%, 35%, 40%, 45%, 50%, 55%, 60%, 65%, 70%, or 75% less than the amount of the redox active compound in the loading dose. In some embodiments, the maintenance dose may be subcutaneously and/or intravenously administered to the subject. In some embodiments, the maintenance dose may administered for 1, 3, or 7 to 10, 12, or 14 day(s) following the intra-arterially administering of the redox active compound. A maintenance dose may be administered 1, 2, 3, or more times per day and/or 1, 2, 3, 4, 5, 6, 7, 10, 12, 14, or more times per week.

A method of the present invention may be carried out prior to, during, and/or after a thrombectomy and/or a cardiac catheterization that is performed on the subject. As used herein, “thrombectomy” refers to the surgical process of removing and/or breakdown of a clot in a subject, regardless of the method used. In some embodiments, a redox active compound of the present invention may be administered concurrently with a thrombectomy and/or a cardiac catheterization that is performed on the subject.

A method of the present invention may further comprise performing a thrombectomy and/or a cardiac catheterization on a subject. In some embodiments, the intra-arterial administration of a redox active compound of the present invention may have an additive effect on a subject when carried out in combination with a thrombectomy and/or a cardiac catheterization. An “additive effect” as used herein refers to the effect of a redox active compound of the present invention and a procedure (e.g., thrombectomy and/or a cardiac catheterization) that is equal to the sum of the effect of the two taken separately. In some embodiments, the intra-arterial administration of a redox active compound of the present invention may have a synergistic effect on a subject when carried out in combination with a thrombectomy and/or a cardiac catheterization.

Synergistic”, “synergy”, or grammatical variants thereof as used herein refer to a combination exhibiting an effect greater than the effect that would be expected from the sum of the effects of the individual components of the combination alone. For example, the terms “synergistic” or “synergy” with regard to a combination of a redox active compound of the present invention and a procedure (e.g., thrombectomy and/or a cardiac catheterization) refers to an efficacy and/or effect (e.g., in treating an ischemia-reperfusion injury in a subject, increasing viability of an organ awaiting transplant, and/or reducing transplant failure in a subject) that is greater than that which would be expected from the sum of the individual effects of the redox active compound and the procedure alone.

A method of the present invention may be carried out prior to, during, and/or after an angioplasty in a subject. As used herein, the term “angioplasty” refers to the surgical process of installing a stent into an artery. In some embodiments, the intra-arterial administration of a redox active compound of the present invention may have an additive effect on the subject when carried out in combination with an angioplasty. In some embodiments, the intra-arterial administration of a redox active compound of the present invention may have a synergistic effect on the subject when carried out in combination with an angioplasty.

A method of the present invention may improve an ischemia-reperfusion injury outcomes in a subject, optionally wherein the outcome is measured in the subject at 1 week after the ischemia-reperfusion injury. In some embodiments, two or more (e.g., 2, 3, 4, 5, or more) outcomes may be improved in the subject. In some embodiments, an improvement in an ischemia-reperfusion injury outcome is a reduction in the number and/or severity of one or more neurological deficit(s), a reduction in the amount of ischemic tissue, and/or a reduction in the infarct volume.

A method of the present invention may improve a neurologic deficit and/or functional recovery after an ischemia-reperfusion injury in a subject, optionally wherein the neurologic deficit and/or ischemia-reperfusion injury functional recovery is measured in the subject at 1 week after the ischemia-reperfusion injury. In some embodiments, the neurological deficit is measured using a neurological examination by a trained professional (e.g., a doctor). In some embodiments, the neurological deficits that may be measured include, but are not limited to, the level of consciousness, the ability to respond to questions and obey commands, the pupillary response, gaze palsy, hemianopsia, dysarthria, limb drift and/or ataxia, sensory loss, aphasia, facial palsy, and/or extinction/inattention of the subject.

A method of the present invention may reduce the amount of ischemic tissue and/or infarct volume caused by an ischemia-reperfusion injury in a subject. In some embodiments, the method may reduce the amount of ischemic tissue in the subject and/or reduce the infarct volume in the subject by about 10%, 20%, 30%, 40%, 50%, 60%, or more. In some embodiments, the method may reduce the amount of ischemic tissue and/or infarct volume in a subject after performing a thrombectomy, embolectomy, angioplasty, and/or a cardiac catheterization in the subject. In some embodiments, the amount of ischemic tissue and/or infarct volume in a subject are measured using a medical imaging technique. In some embodiments, the medical imaging technique is magnetic resonance imaging (MRI).

In some embodiments, the ischemia-reperfusion injury may be caused by an ischemic stroke, a heart attack, and/or an organ transplant. In some embodiments, the organ transplant may cause an ischemia-reperfusion injury in the subject and/or in the transplanted organ.

In some embodiments, a redox active compound of the present invention may be intra-arterially administered to a subject within about 1, 2, 3, 4, 5, 6, 7, 8, 9, 10, 11, or 12 hours of an ischemia-reperfusion injury within said subject. In some embodiments, the ischemia-reperfusion injury is a stroke, heart attack, or organ transplant in the subject. In some embodiments, a second dosage of a redox active compound of the present invention may be administered to a subject 1 day or more (e.g., 2, 3, 4, 5, 6, 7, 8, or more days) after the intra-arterial administration of the redox active compound.

A method of the present invention may increase viability of an organ awaiting transplant and/or reduce transplant failure in a subject, the method comprising contacting the organ awaiting transplant and a redox active compound of the present invention. As used herein, “contact,” “contacting,” “contacted,” and grammatical variations thereof, in reference to a redox active compound of the present invention and an organ awaiting transplant refer to placing the redox active compound and organ together under conditions suitable for allowing the redox active compound to contact and/or enter the organ and/or a cell thereof. In some embodiments, the contacting comprises contacting a composition comprising the redox active compound and the organ. The composition may be a transplant medium. In some embodiments, the contacting comprises incubating (e.g., placing and/or submerging) and/or culturing the organ in a composition comprising the redox active compound (e.g., in a transplant media comprising the redox active compound). In some embodiments, the contacting comprises perfusing the organ awaiting transplant with a composition comprising the redox active compound. In some embodiments, a composition comprising the redox active compound may include the redox active compound in a concentration of about 0.1, 0.5, 1, 2, or 5 mg/mL to about 10, 15, 20, or 25 mg/mL. In some embodiments, the method increases viability of an organ awaiting transplant by about 1, 2, 5, or 10 hour(s) to about 15, 20, 30, 40, 50, 60, or 72 hours. In some embodiments, the contacting occurs prior to transplanting the organ awaiting transplant into the subject and the method comprises transplanting the organ awaiting transplant into the subject. In some embodiments, after transplanting the organ awaiting transplant into the subject, the method further comprises administering a redox active compound of the present invention to the subject and/or organ.

The present invention is explained in greater detail in the following non-limiting examples.

### EXAMPLES

### Example 1

### Background

Stroke is a leading cause of death and disability in the United States. Nearly 800,000 people suffer from this disease each year. 87% of these patients have a stroke that is ischemic in nature.¹ When a cerebral artery is blocked by blood clot, it immediately leads to neurologic and functional deficits. The brain tissue within the arterial territory is metabolically compromised at the beginning, but is still salvageable if reperfusion is achieved within a short period of time.² Early restoration of blood flow is critical for the treatment of stroke patients. Intravenous tissue plasminogen activator (t-PA) was approved by FDA as a standard thrombolytic therapy.³ Endovascular mechanical thrombectomy also became popular as an effective recanalization procedure⁴⁻¹⁰, significantly improving the clinical outcome. However, there are some shortcomings of these treatments, including the narrow therapeutic time window of t-PA of 4.5 hours after stroke onset, which limits its use to <10% of patients.^{11,12} More than 50% of thrombectomy patients still do not gain functional independence.^{13,14}

Manganese (III) ortho isomeric N-substituted pyridylporphyrins (MnPs) are a class of potent redox-active compounds, commonly known as mimics of superoxide dismutase family of enzymes.¹⁵⁻¹⁸ These compounds are able to interact with other reactive species also, such as peroxynitrite, hydrogen peroxide (H₂O₂) and nitric oxide (NO). Yet, the ability of MnPs to oxidize/S-glutathionylate cysteines of signaling proteins appears to be their major mode of action. Thus, MnPs employ H₂O₂/glutathione to catalyze oxidation/S-glutathionylation of cysteines of numerous proteins, including NF-κB and Nrf2/Keap1 transcription factors, thereby modifying their activity and in turn affecting proliferative and apoptotic pathways. 16.19.20 Inhibition of NF-κB, while activation of Nrf2 by MnPs, with subsequent upregulation of endogenous antioxidant enzymes (such as Mn superoxide dismutase, catalase and peroxiredoxins), plays critical role in protection of normal tissue.¹⁹ This study aimed to investigate the long-term effect of intracarotid BMX-001 infusion in a rat model of ischemic stroke. Since, in a clinical scenario, a catheter is already in place in thrombectomy patients, this manganese porphyrin was investigated for its ability enhance stroke recovery as an adjunct to thrombectomy.

### Materials and Methods

The care and handling of the animals were in accordance with the National Institutes of Health guidelines. Male Wistar rats (Hsd: WI, 250-275 grams) were purchased from Envigo (Indianapolis, IN, USA) and housed at the Duke vivarium with free access to food and water and a 12 hours light/12 hours dark cycle. Room temperature and humidity were well controlled. A GMP-grade batch of BMX-001 was used for the studies.

### Ischemic Stroke Model

The rats were fasted overnight for glucose control. On the surgery day, they were anesthetized with 5% isoflurane in 30% oxygen balanced with nitrogen. The trachea was orally intubated, and both lungs were mechanically ventilated to maintain normocapnia. Isoflurane was reduced to 1.5% during the surgical procedure. A 22-gauge needle thermistor was percutaneously inserted beneath the temporalis muscle adjacent to the skull. The pericranial temperature was maintained at 37.0° C.±0.2° C. by a surface heat lamp. The tail artery was cannulated with PE 50 tubing (BD Intramedic™ Polyethylene Tubing, Sparks, MD, USA), and arterial blood pressure was continuously monitored. 50 IU of heparin was given via arterial line to prevent intra-arterial thrombosis. Arterial blood samples were collected pre, during, and after ischemia for blood gas, glucose, and hematocrit measurements.

Ischemic stroke was induced by transiently occluding the middle cerebral artery (MCAO) as previously reported.^{23,24} Briefly, a midline cervical incision was made and the right common carotid artery (ECA) was identified. The external and internal carotid arteries were dissected. The distal external carotid artery and its superior thyroid branch were isolated, ligated and divided. The internal carotid artery (ICA) was dissected distally until the origin of the pterygopalatine artery was visualized. After surgical preparation, a 10 min interval was allowed for physiologic stabilization. Then, the internal and common carotid arteries were temporally blocked, a small incision was cut on the ECA stump, and a silicone coated 4-0 nylon monofilament (Doccol MCAO suture, 403745) was inserted through the ECA and passed distally into the ICA until a slight resistance was felt, and then secured using silk suture. The common carotid artery was re-opened, and a timer was started as the onset of cerebral ischemia.

After 90 min of MCAO, the filament was removed and a PE-10 tube (BD Intramedic™ Polyethylene Tubing, Sparks, MD, USA) was inserted into the ICA beyond the origin of the pterygopalatine artery. The vehicle or BMX-001 (30-50 μg/kg) solution (1 μl/g for both solutions, 250 μl for the 250 g rat) was slowly infused for 5 minutes via syringe pump. At the completion of intra-carotid infusion, the PE tube was removed and the ECA stump was permanently ligated. The tail artery catheter was removed and both the neck incision and tail incision were infiltrated with bupivacaine and closed with suture. Isoflurane was discontinued. Rats were disconnected from the ventilator when they attained spontaneous respiration and kept in a recovery cage. The tracheas were extubated after recovery of the righting reflex. They were in an O₂ enriched environment (30% O₂) for 1 h and then returned to the home cage.

### Neurological Score

Post-stroke neurologic deficits were assessed at day 7 and 28 using a standardized neurological scoring system.^{27,28} General status, simple motor deficit, complex motor deficit, and sensory deficit were evaluated, including the ability of walking on a beam and climbing on the vertical screen. The total score each animal received was the sum of all four individual scores (0-48, 0=normal and 48=the worst score). Such scoring system has been shown to correlate well with magnitude of cerebral infarct volume.²⁷

### Cerebral Infarct Volume Measurement

Upon the completion of functional tests, the animals were weighed, anesthetized with isoflurane, and decapitated. The brains were harvested, frozen at −20° C. in 2-methylbutane on dry ice, and stored in a −80° C. freezer. Serial quadruplicate 20-μm-thick coronal sections were cut using a Leica cryostat and mounted on the slides at 720-μm interval over the rostral-caudal extent of the brain. The sections were dried on a slide heater at 36° C., and stained with hematoxylin and eosin. A section from each 800-μm interval was digitized with a video camera controlled by an image analyzer (MCID, the MicroComputer Imaging Device, Imaging Research Inc, St. Catharines, Ontario, Canada). The image of each section was stored as a 1280×960 pixel matrix and displayed on a computer monitor. With the observer blinded to the experimental condition, the following regions of interest (ROI) were outlined individually using an operator-controlled cursor, including non-infarcted ipsilateral cerebral cortex, non-infarcted ipsilateral subcortex, contralateral cerebral cortex, and contralateral subcortex. The area in each ROI (mm²) was determined by automated counting of calibrated pixels and the volume (mm³) was computed by the known interval between sections over the rostral-caudal extent of brain. Ischemic tissue volume was calculated by subtracting ipsilateral non-infarcted tissue volume from the corresponding contralateral tissue volume.

### Experiment 1: Effect of BMX-001 on Acute Stroke Outcome in Young Male Rats

30 young male Wistar rats (250-275 grams) were subjected to 90 min of MCAO and then randomly assigned to vehicle (n=16) or BMX-001 treatment group (n=14). The 30 μg/kg bolus dose of BMX-001 or same amount of saline was given through the carotid artery immediately after the reperfusion. That was followed by subcutaneous injection of vehicle or BMX-001 at 225 μg/kg twice per day for 7 days. Animals were weighed and fed with soft food daily. Neurological deficits and infarct volumes were examined at day 7 post-stroke.

### Experiment 2: Effect of BMX-001 on Long-Term Stroke Outcome in Young Female Rats

30 young female Wistar rats (225-250 grams) were subjected to 90 min of MCAO and randomly assigned to the vehicle (n=15) or BMX-001 treatment group (n=15). Rats received 7 days of treatment as described in Experiment 1. Animals were weighed and fed with soft food daily during the first week. Neurologic scores were evaluated at day 7 and 28. Infarct volumes were measured at 28 days.

### Experiment 3: Effect of the Duration of the Treatment of BMX-001 on Long-Term Stroke Outcome in Young Male Rats

57 young male Wistar rats (250-275 grams) were subjected to 90 min of MCAO and then randomly assigned to the vehicle (n=19), 50 μg/kg intra-carotid bolus of BMX-001 followed by 7 days of subcutaneous doses at 225 μg/kg twice per day (n=19), or 50 μg/kg intra-carotid bolus of BMX-001 followed by 28 days of subcutaneous doses at 225 μg/kg twice per day (n=19). The BMX-001 group that received bolus intra-carotid infusion and 7 days of maintenance doses, also received 21 days of subcutaneous vehicle injections to allow for valid comparison to the group that was injected subcutaneously with BMX-001 for 28 days. Animals were weighed and fed with soft food daily during the first week. Neurologic scores were evaluated at 7 and 28 days. Infarct volumes were measured at 28 days.

### Experiment 4: Effects of BMX-001 on Stroke Outcome in Young Spontaneously Hypertensive Rats

30 young male spontaneously hypertensive rats (SHR rats, 225-250 grams) were subjected to 90 min of MCAO, and randomly assigned to the vehicle (n=15) or BMX-001 treatment (n=15) group. Post-stroke treatment comprised intracarotid bolus of 50 μg/kg BMX-001 or vehicle plus 7 days of 225 μg/kg BMX-001 subcutaneous injection twice per day. Animals were weighed and fed with soft food daily during the first week. Neurologic scores were evaluated at 7 and 28 days. Infarct volumes were measured at 28 days.

### Experiment 5: Effect of BMX-001 on Stroke Outcome in Aged Female Rats

34 aged female Fisher 344 rats (220-250 grams) were subjected to 90 min of MCAO and randomly assigned to the vehicle (n=17) or BMX-001 (n=17) group. Treatment and outcome measures are the same as in Experiment 4.

### Experiment 6: BMX-001Plasma and Brain Concentration Measurement

Young male Wistar rats (n=4) were subjected to 90 min of MCAO and 30 μg/kg of BMX-001 intra-carotid infusion following reperfusion. Blood and brain samples were harvested at 30 min after BMX-001 infusion, stored in −80° C. freezer, and measured using Liquid chromatography-tandem-mass spectroscopy (LC/MS/MS) analysis. Brain tissue was homogenized in water (1 g in 2 mL water). Then, 150 mL homogenate or 50 mL plasma, two Zr-silica beads (2.5 mm), 150 mL (for brain tissue) or 50 mL (for plasma) 1% HFBA (heptafluorobutyric acid) in 30% acetonitrile/water+BMX-001-d8 (int. std.,50 nM), and 600 mL (for brain) or 200 mL (for plasma) of chloroform/isopropanol (4/1, v/v) was added to polypropylene tube and vigorously agitated in FastPrep (Thermo Savant) at speed 6 for 45 s, followed by centrifugation at 16,000 rcf for 5 min at room temperature. The 450 mL (for brain) or 150 mL (for plasma) aliquot of organic (lower) layer was transferred to polypropylene tube and evaporated to dryness by gentle stream of nitrogen. The dry residue was reconstituted into 50 mL of mobile phase A, and 20 mL injected into LC/MS/MS system. The LC/MS/MS analysis was performed on Agilent 1200 HPLC-AB/SCIEX 5500 QTrap MS/MS instrument. LC column: Phenomenex Kinetex 4×3 mm, C18 guard cartridge at 35° C.; mobile phase A: 0.05% HFBA, 10% acetonitrile, water; mobile phase B: 0.05% HFBA in acetonitrile; elution gradient: 0-1 min 0-70% B, 1-2 min 70% B, 2-2.1 min 70-100% B, 2.1-2.6 min 100% B, 2.6-2.7 min 100-0% B; run time: 4 min. MS/MS transitions: BMX-001 (3×HFBA) at m/z 857.3/599.7. Calibration samples in 2.4 (LOQ)—300 nM brain tissue or 0.47 (LOQ)—30 nM plasma concentration range were prepared by adding known amounts of BMX-001 into blank brain homogenate or plasma, and analyzed alongside study samples as a single analytical batch.

### Experiment 7: Effect of BMX-001 Infused Via Carotid Artery on Blood Brain Barrier Permeability and Vascular Endothelial Wall

30 μg/kg of BMX-001 (n=4), or same amount of saline (n=4) or mannitol (n=4) was given to normal male Wistar rats through the carotid artery. The rats received 100 μl 2% Evans blue intravenous infusion 30 min later and then intracardially perfused using 100 ml normal saline at 30 min following infusion. Brains were harvested and the Evans blue content was measured spectrophotometrically.

30 μg/kg of BMX-001 (n=4), or same amount of saline (n=4) or Solu-Medrol (n=4) was given to normal Wistar rats through the carotid artery. Rats were recovered for 3 days and perfused intracardially with normal saline followed by 10% formalin. Brains were paraffin embedded, cut, and stained with hematoxylin and eosin. Vascular histologic damage was assessed.

### Experiment 8. Interaction of BMX-001 with t-PA

Fresh arterial blood samples were collected from young male rats and distributed into 21 pre-weighed centrifuge tubes. After sitting at 37° C. for 3 hours, the blood clots were formed. The supernatant was removed from these tubes and then the tubes with blood clots were weighed. The weight of the blood clot was calculated (=the tube with blood clot-pre-weighed tube). Then, 1 ml saline, t-PA (1 mg/ml) or t-PA (1 mg/ml) plus BMX-001 (50 μg/ml) were added (n=7 for each group) and tubes placed in 37° C. dry incubator for 3 hours. The liquids were then removed from the tubes. The tubes with remaining blood clots were weighed again. The percentage of blood clot dissolved by t-PA was calculated.

All outcome measures were performed by trained laboratory personnel who were blinded to group assignment.

### Statistical Analysis

Most of the data were expressed as mean±SD and analyzed using unpaired student t-test or one-way ANOVA for more than two groups. Neurologic scores are nonparametric, expressed as median±IQR, and analyzed using the Mann-Whitney test. All statistical analyses were performed using Prism 6 software (GraphPad Software Inc, San Diego, CA, USA), and a p-value <0.05 was considered statistically significant.

### Results

### BMX-001 Provided Acute Protection in Young Male Stroke Rats

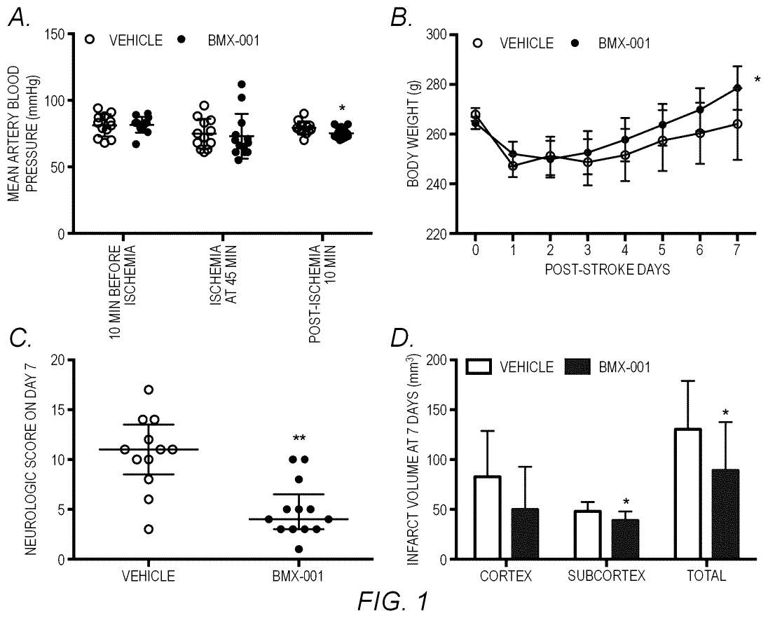

30 young male rats were subjected to 90 min of MCAO. One died during surgery due to a bleeding from the carotid artery and one vehicle rat died 24 hours after stroke. Intracranial hemorrhage was not found. Three rats, including 2 vehicle rats and one BMX-001 rat, did not circle to the left after recovering from anesthesia. Thus, 5 rats were excluded from this study. The 12 vehicles and 13 BMX-001 rats were analyzed for outcomes. Perioperative blood pressures (10 minutes before ischemia, 45 min ischemia, and 10 minutes post-ischemia) were 81±8 mmHg, 75 ±11 mmHg, and 79±5 mmHg in the vehicle and 82±6 mmHg, 73±16 mmHg, and 75±4 mmHg in BMX-001 group. No intra-group blood pressure difference was found at 10 min before the ischemia and 45 min after the onset of ischemia. However, we found that the blood pressure was slightly dropped after BMX-001 treatment (79±5 mmHg in the vehicle and 75±4 in BMX-001, p=0.04, FIG. 1, Panel A). Animals in both groups had a significant body weight loss at 24 hours post-stroke (247±15 grams in vehicle, p<0.001 vs baseline 267±9 grams, and 252±9 grams in BMX-001, p<0.001 vs baseline 265±9 grams). Body weight loss was recovered slowly in vehicle treated rats (264±14 at day 7, p=0.278 vs 247±15 grams at 24 hours). BMX-001 treatment promoted body weight recovery (279±9 grams at day 7, p=0.02 vs 252±9 grams at 24 hours, FIG. 1, Panel B).

Neurologic scores at post-stroke day 7 were 11±5 in vehicle and 4±3.5 in BMX-001 treated animals (p<0.01, FIG. 1, Panel C). Vehicle group had a large brain infarct (total infarct 130.354±48.591 mm3, cortex infarct 82.670±46.134 mm³, and subcortex infarct 48.108±9.256 mm³) and BMX-001 daily treatment reduced the infarct size (total infarct 89.097±48.424 mm³, p =0.04 vs vehicle; cortex infarct 49.864±42.899 mm³, p=0.07 vs vehicle; subcortex infarct 38.964 ±8.892 mm³, p=0.02 vs vehicle, FIG. 1, Panel D).

### BMX-001 Reduced Neurologic Deficit in Long-Term Survived Young Female Rats

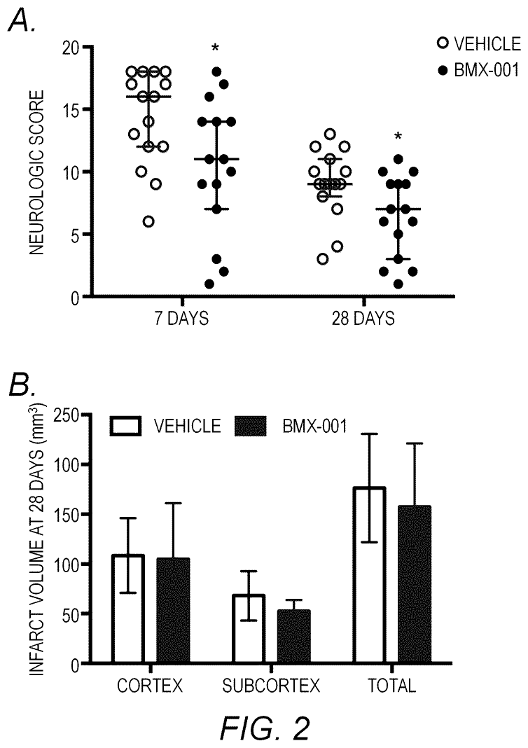

30 female rats were subjected to 90 min of MCAO and all of them survived for 28 days. Although these female rats were at the age same as male, their size appeared small. We used the same size of filament for both male and female rats, that induced more severe ischemia in female rats compared to male rats, probably due to the small ICA diameter in the female rats. Neurologic scores at 7 days were 16±6 in female vehicle, and 11±5 in male rats. BMX-001 treatment improved post-stroke neurologic deficit at both 7 days and 28 days (FIG. 2, Panel A, 7 days, 16±6 in vehicle group, 11±7 in BMX-001 group, p=0.04; 28 days, 9±3 in vehicle group, 7±6 in BMX-001 group, p=0.04). Although BMX-001 treated rats had small infarct volume compared to vehicle, there was no significant difference detected between groups (FIG. 2, Panel B, 157.378 ±63.699 mm³ in BMX-001 group vs 176.334±54.432 mm³ in vehicle group, p=0.38). While not wishing to be bound to any particular theory, it seems that the doses of BMX-001 in this experiment were not sufficient for rescuing brain tissue that underwent severe ischemia.

### BMX-001 Treatment for 7 Days Provides Long-Term Protection in Young Male Rats

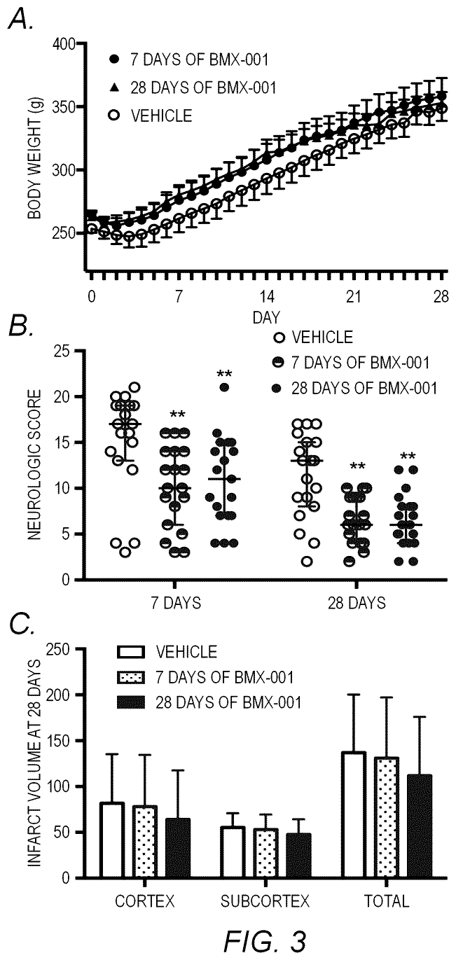

57 rats were subjected to 90 min of MCAO and perioperative physiological parameters, including blood pressure, blood gas, glucose and hematocrit, were controlled at similar levels (Table 1). No intra-group difference was observed. All rats recovered from surgery, received treatments and survived for 28 days. Vehicle rats had a slower body weight recovery (FIG. 3, Panel A, body weight on day 7 post-being 261±47 grams in vehicle group, 276±46 grams on day 7 in BMX-001 group, and 280±33 grams on day 28 in BMX-001 group, p=0.37). BMX-001 treatment promoted post-stroke functional recovery and improved long-term functional recovery. Neurologic scores were improved on day 7 (FIG. 3, Panel B, 17±6 in vehicle group, 10±8 in 7 days of BMX-001 group, and 11±8 in 28 days of BMX-001 group, p=0.0042), and day 28 (FIG. 3, Panel B, 13±7 in the vehicle, 6±5 in 7 days of BMX-001, and 6±4 in 28 days of BMX-001, p=0.0012). The vehicle group had a larger total infarct (FIG. 3, Panel C, 137±63 mm³ in the vehicle group, 131±66 in 7 days of BMX-001 group, and 112±64 on day 28 in BMX-001 group, p=0.46). While not wishing to be bound to any particular theory, the data in this experiment did not prove further functional improvement by treatment beyond one week and suggest that one week of BMX-001 treatment is sufficient for obtaining long-term protection.

TABLE 1
Perioperative Physiologic Values (mean ± SD) in young male Wistar rats

| Time of | | | BMX-001 for 7 | BMX-001 for |
|---|---|---|---|---|
| measurement | Measurement | Vehicle (n = 9) | days (n = 19) | 28 days (n = 19) |
| 10 minutes | MAP (mmHg) | 76 ± 7 | 75 ± 9 | 75 ± 7 |
| before stroke | pH | 7.476 ± 0.034 | 7.481 ± 0.033 | 7.487 ± 0.039 |
| | PCO₂ | 36 ± 5 | 36 ± 3 | 35 ± 4 |
| | PO₂ | 157 ± 32 | 168 ± 18 | 172 ± 13 |
| | Glucose | 109 ± 17 | 104 ± 19 | 110 ± 17 |
| | Hct | 42.6 ± 1.6 | 42.6 ± 1.4 | 42.2 ± 1.3 |
| 45 minutes after | MAP (mmHg) | 72 ± 9 | 69 ± 7 | 69 ± 7 |
| stroke onset | pH | 7.450 ± 0.035 | 7.462 ± 0.048 | 7.433 ± 0.049 |
| | PCO₂ | 38 ± 4 | 37 ± 6 | 39 ± 6 |
| | PO₂ | 162 ± 21 | 162 ± 17 | 162 ± 17 |
| 10 minutes post-stroke | MAP (mmHg) | 71 ± 6 | 67 ± 5 | 71 ± 6 |

MAP, mean blood pressure; SD, standard deviation; PCO2, partial pressure of carbon dioxide; PO2, partial pressure of oxygen; Hct, hematocrit.

### BMX-001 Improved Post-Stroke Long-Term Functional Deficits in Spontaneously Hypertensive Rats

30 young male, spontaneously hypertensive rats were subjected to 90 min ofMCAO. Their mean arterial blood pressure was high in both groups before stroke (127±16 mmHg in the vehicle and 124±21 in BMX-001). For comparison, the blood pressure in young male Wistar rats was around 80 mmHg. Perioperative physiological parameters, including blood pressure, blood gas, glucose and hematocrit, were at similar levels in both groups (Table 2). All rats recovered from surgery. However, four rats died after one week (one vehicle at post-stroke 10 days, three BMX-001 rats at 8, 9 and 10 days after stroke, respectively). At the completion of the study, 14 vehicles and 12 BMX-001 rats survived for 28 days.

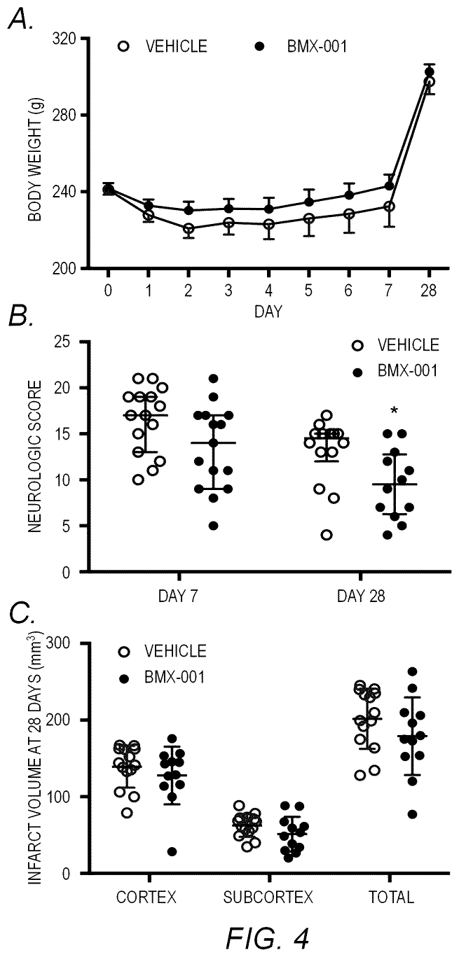

Vehicle rats had a slow body weight recovery (FIG. 4, Panel A). The neurologic score was 17±6 on day 7 and 14.5±3 on day 28 (FIG. 4, Panel B). BMX-001 improved neurologic score (14±8 at day 7, p=0.05 vs vehicle; 9.5±6.25 at day 28, p=0.018 vs vehicle, Mann-Whitney test). The infarct volumes were smaller in BMX-001 group (FIG. 4, Panel C, total infarct volume, 202±39 mm³ in vehicle group, 179±51 in BMX-001 group, p=0.21; cortex infarct volume, 139 ±27 mm³ in vehicle group, 128±37 in BMX-001 group, p=0.37; subcortex infarct volume, 63 ±15 mm³ in vehicle, 51±23 in BMX-001, p=0.14).

TABLE 2
Perioperative Physiologic Values
(mean ± SD) in young male SHR rats

| Time of | | Vehicle | BMX-001 | P- |
|---|---|---|---|---|
| measurement | Measurement | (n = 15) | (n = 15) | value |
| 10 minutes | MAP (mmHg) | 127 ± 16 | 124 ± 20 | 0.58 |
| before stroke | pH | 7.402 ± 0.023 | 7.384 ± 0.046 | 0.19 |
| | PCO₂ | 39 ± 3 | 41 ± 6 | 0.38 |
| | PO₂ | 173 ± 24 | 175 ± 18 | 0.72 |
| | Glucose | 97 ± 31 | 93 ± 30 | 0.71 |
| | Hct | 48 ± 3 | 48 ± 3 | 0.76 |
| 45 minutes | MAP (mmHg) | 91 ± 13 | 90 ± 9 | 0.93 |
| after stroke | pH | 7.379 ± 0.027 | 7.375 ± 0.038 | 0.71 |
| onset | PCO₂ | 42 ± 4 | 42 ± 5 | 0.83 |
| | PO₂ | 170 ± 16 | 175 ± 15 | 0.37 |
| 10 minutes | MAP (mmHg) | 89 ± 9 | 83 ± 11 | 0.15 |
| post-stroke | | | | |

SHR, spontaneous hypertension rats; MAP, mean blood pressure; SD, standard deviation; PCO2, partial pressure of carbon dioxide; PO2, partial pressure of oxygen; Hct, hematocrit.

### BMX-001 Improved Long-Term Stroke Outcome in Aged Female Rats

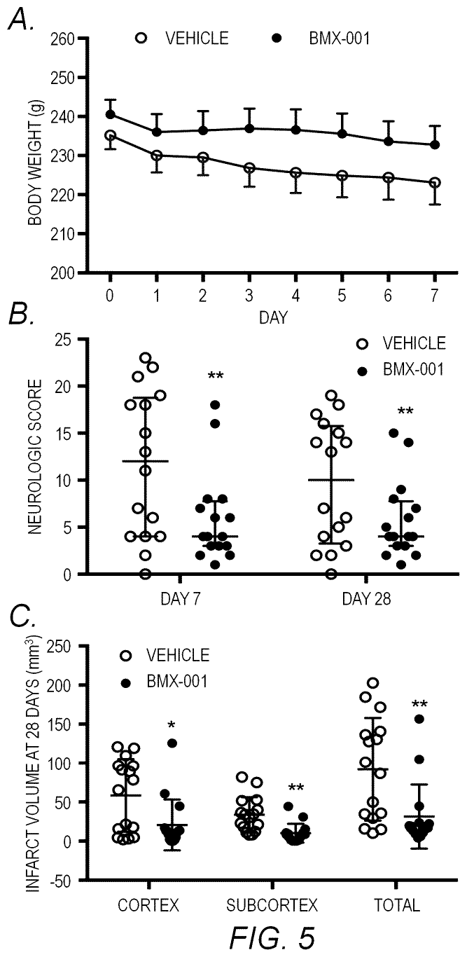

34 aged rats were subjected to 90 min of MCAO and recovered from surgery. Two rats died at post-stroke day 2 due to large infarct, including one vehicle rat and one BMX-001 treated rat. Rats were randomly assigned to the treatment groups after surgery and BMX-001 group had slightly higher blood pressure before stroke (114±11 mmHg) compared to the vehicle (105±17 mmHg, p=0.06). Other physiological parameters were similar (Table 3). There was a different body weight recovery pattern between aged and young rats. Within the first week post-stroke, all rats continued to lose body weight although soft food was provided (FIG. 5, Panel A). Young rats usually began to gain weight from the third day post-stroke. Body weight was 223±22 grams on day 7 post-stroke in the vehicle, a 12 grams loss from the baseline body weight (235±14 grams). BMX-001 treated group had a body weight of 233±19 grams on day 7 post-stroke, i.e., the 8 grams loss from the baseline body weight (241±15 grams). Such data indicated that general status was improved by BMX-001 treatment. Vehicle rats had more severe neurologic deficits compared to BMX-001 treatment on both day 7 and day 28 post-stroke (FIG. 5, Panel B). Neurologic scores in the vehicle were 12±14.75 on day 7, and 10±12.5 on 28 days compared to 4±4.75 on day 7 and 4±4.75 on day 28 in BMX-001 (p<0.001). Infarct volume was significantly reduced after BMX-001 treatment as well. The total infarct volume was 92±65 mm³ in the vehicle rats, including cortex infarct volume 58.5±46 mm³ and subcortex infarct volume 33.5±11 mm³ (FIG. 5, Panel C). BMX-001 induced a 60% reduction of infarct volume (total infarct volume 31±41 mm³, p=0.0037 vs vehicle; cortex infarct volume 21±32 mm³, p=0.0122 vs vehicle; subcortex infarct volume 10±12 mm³, p=0.0010).

TABLE 3
Perioperative Physiologic Values
(mean ± SD) in aged female Fisher 344 rats

| Time of | | Vehicle | BMX-001 | P- |
|---|---|---|---|---|
| measurement | Measurement | (n = 17) | (n = 17) | value |
| 10 minutes | MAP (mmHg) | 105 ± 17 | 114 ± 11 | 0.06 |
| before stroke | pH | 7.473 ± 0.061 | 7.453 ± 0.045 | 0.31 |
| | PCO₂ | 33 ± 5 | 33 ± 4 | 0.58 |
| | PO₂ | 143 ± 39 | 142 ± 19 | 0.37 |
| | Glucose | 112 ± 35 | 103 ± 28 | 0.40 |
| | Hct | 41 ± 6 | 43 ± 3 | 0.15 |
| 45 minutes | MAP (mmHg) | 91 ± 14 | 98 ± 14 | 0.14 |
| after stroke | pH | 7.425 ± 0.047 | 7.402 ± 0.060 | 0.27 |
| onset | PCO₂ | 37 ± 5 | 40 ± 8 | 0.10 |
| | PO₂ | 150 ± 25 | 146 ± 20 | 0.63 |
| 10 minutes | MAP (mmHg) | 89 ± 12 | 90 ± 13 | 0.62 |
| post-stroke | | | | |

MAP, mean blood pressure; SD, standard deviation; PCO2, partial pressure of carbon dioxide; PO2, partial pressure of oxygen; Hct, hematocrit.

### BMX-001 Accumulated at the Site of Brain Injury

Four rats were subjected to 90 min of MCAO and then 30 μg/kg of BMX-001 was given through the carotid artery immediately following reperfusion. Blood and brains were harvested 30 min later. BMX-001 contents were 11.3±1.2 nM in plasma, 7.4±8.6 nM in ipsilateral brain hemisphere, and 0.5±0.3 nM incontralateral brain hemisphere. Direct intracarotid infusion led to a 15-fold greater BMX-001 concentration at the site of brain injury than in the contralateral hemisphere.

### Intracarotid BMX-001 Infusion Did not Affect the Blood Brain Barrier Permeability and Vascular Structure

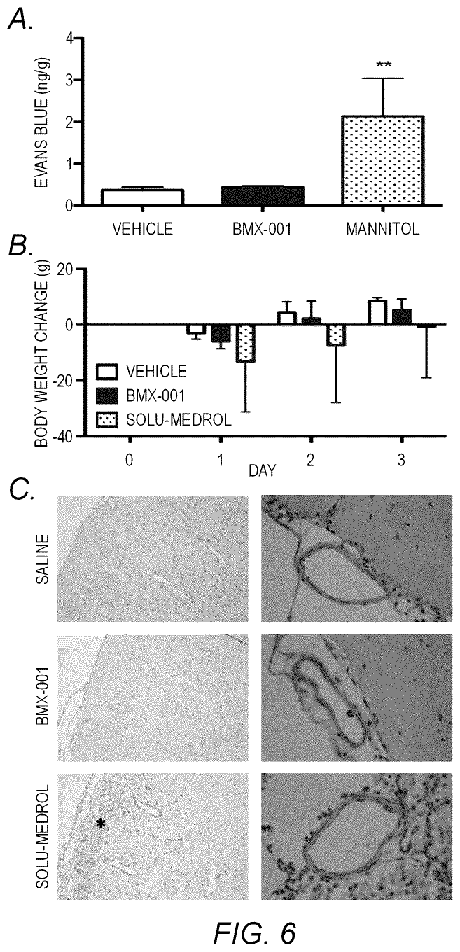

After intracarotid infusion of vehicle, BMX-001 and mannitol, we injected 0.1 ml of 2% Evans blue through the jugular vein in 12 non-stroke rats. Blood was flushed using normal saline 30 minutes later and then brains were harvested for Evans blue content measurement. Mannitol serves as positive control; a significant increase of Evans blue content was found in the Mannitol group (FIG. 6, Panel A). BMX-001 group had only somewhat higher Evans blue content, but an intra-group difference was not detected between the vehicle and BMX-001 groups.

An additional 12 non-stroke rats were used for brain histology. Solu-Medrol served as a positive control. All rats had a body weight decrease on day 1 after intracarotid infusion (FIG. 6, Panel B) and then recovered on day 2 and day 3. The solu-Medrol group is the only group that had not recovered to the baseline body weight even at 72 hours after infusion. Brain histology demonstrated that there was brain damage in the rat who received Solu-Medrol. The brain tissues and vessels were normal in the rats treated with vehicle and BMX-001 (FIG. 6, Panel C).

BMX-001 does not React with t-PA

When the blood clots were incubated with saline (vehicle), 1 mg/ml t-PA, or 1 mg/ml t-PA plus 50 μg/ml BMX-001 at 37° C. for 30 minutes, the blood clots were changed to 0.14±4.05%, 44.77±25.77%, and 43.23±24.88%, respectively. Saline had a negligible effect on blood clots, but a significant dissolution of blood clots was seen with t-PA or t-PA plus BMX-001. There was no difference between t-PA and t-PA plus BMX-001.

### DISCUSSION

Extensive clinical trials demonstrated that endovascular thrombectomy is an effective way to restore the blood flow in selected stroke patients and improved their clinical outcome.^{14,29-31} The endovascular thrombectomy was reportedly associated with significantly higher rates of angiographic revascularization at 24 hours compared with standard medical care (75.8* vs 34.1%; OR, 6.49; 95% CI, 4.79-8.79; P<0.001).²⁹ In the patients with stroke in the proximal anterior intracranial circulation, thrombectomy with a stent retriever within 6 hours after onset plus intravenous t-PA reduced disability at 90 days over the entire range of scores on the modified Rankin scale (P<0.001).³² Currently, the time window for thrombectomy has been extended to 24 hours.⁹ However, some thrombectomy patients did not recover their functional independence. Therefore, there is an immediate need for the new therapies adjunct to thrombectomy. In the clinical trial of patients with acute ischemic stroke caused by a proximal intracranial arterial occlusion, the intra-arterial treatment with t-PA within 6 hours after onset of symptoms is effective and safe for revascularization.³³ Thus, intra-arterial fibrinolytics have been utilized as adjunct to mechanical thrombectomy to further improve the reperfusion.³⁴⁻³⁶ An ortho isomeric Mn N-ethylpyridylporphyrin, MnTE-2-PyP⁵⁺ (AEOL10113, BMX-010), provided protection in an animal model of ischemic stroke even when given 6 hours after reperfusion.²¹ This time window was found in other two MnP analogs as well.^{23,24} We have evaluated here the therapeutic potential of a 3^{rd} generation Mn porphyrin, MnTnBuOE-2-PyP⁵⁺ (BMX-001) as an adjunct to mechanical thrombectomy.

In this study, BMX-001 significantly improved the neurologic deficits and reduced infarct size in young male Wistar rats when assessed at 7 days post-stroke. Long-term experiments also demonstrated its protective effects on neurologic functional improvement in female, SHR and aged rats. Moreover, the intra-carotid infusion of BMX-001 did not affect the BBB permeability and vascular structure. Further, BMX-001 did not react with t-PA. Therefore, BMX-001 appears to be a safe adjunct to thrombectomy and/or t-PA treatment.

The infarct volume in long-term experiments was calculated by subtracting the remaining ipsilateral brain tissue from the volume of the contralateral hemisphere. At four weeks post-stroke, the infarct area was completely destroyed and absorbed. The non-ischemia area also shrank due to neuronal degeneration and loss of neuronal innervation derived from the ischemic area. While not wishing to be bound by any particular theory, the problems we encountered are entirely different from the histological outcome assessed at 7 days post-stroke and are therefore the likely cause for the lack of the effect of BMX-001 on infarct volume in long-term experiments on young male, female and SHR rats.

### CONCLUSIONS

BMX-001 is an experimental Mn porphyrin-based drug currently in use in several Phase II clinical trials. In this work, we demonstrated that intracarotid infusion, immediately following reperfusion, improved post-stroke neurologic deficits in a rat model of cerebral ischemia regardless of the gender, age, and hypertension of the animals.

### REFERENCES

1. Benjamin E J, Virani S S, Callaway C W, Chamberlain A M, Chang A R, Cheng S, Chiuve S E, Cushman M, Delling F N, Deo R, et al. Heart Disease and Stroke Statistics-2018 Update: A Report From the American Heart Association. Circulation. 2018; 137: e67-e492
2. Astrup J, Siesjo B K, Symon L. Thresholds in cerebral ischemia—the ischemic penumbra. Stroke. 1981; 12:723-725
3. National Institute of Neurological D, Stroke rt PASSG. Tissue plasminogen activator for acute ischemic stroke. N Engl J Med. 1995; 333:1581-1587
4. Jovin T G, Nogueira R G, Lansberg M G, Demchuk A M, Martins S O, Mocco J, Ribo M, Jadhav A P, Ortega-Gutierrez S, Hill M D, et al. Thrombectomy for anterior circulation stroke beyond 6 h from time last known well (AURORA): a systematic review and individual patient data meta-analysis. Lancet. 2022; 399:249-258
5. Jovin T G, Chamorro A, Cobo E, de Miquel M A, Molina C A, Rovira A, San Roman L, Serena J, Abilleira S, Ribo M, et al. Thrombectomy within 8 Hours after Symptom Onset in Ischemic Stroke. New Engl J Med. 2015; 372:2296-2306
6. Campbell B C V, Donnan G A, Mitchell P J, Davis S M. Endovascular thrombectomy for stroke: current best practice and future goals. Stroke Vasc Neurol. 2016; 1:16-22
7. Bracard S, Ducrocq X, Mas J L, Soudant M, Oppenheim C, Moulin T, Guillemin F, investigators T. Mechanical thrombectomy after intravenous alteplase versus alteplase alone after stroke (THRACE): a randomised controlled trial. Lancet Neurol. 2016; 15:1138-1147
8. Saver J L, Chapot R, Agid R, Hassan A, Jadhav A P, Liebeskind D S, Lobotesis K, Meila D, Meyer L, Raphaeli G, et al. Thrombectomy for Distal, Medium Vessel Occlusions: A Consensus Statement on Present Knowledge and Promising Directions. Stroke. 2020; 51:2872-2884
9. Nogueira R G, Jadhav A P, Haussen D C, Bonafe A, Budzik R F, Bhuva P, Yavagal D R, Ribo M, Cognard C, Hanel R A, et al. Thrombectomy 6 to 24 Hours after Stroke with a Mismatch between Deficit and Infarct. N Engl J Med. 2018; 378:11-21
10. Albers G W, Lansberg M G, Brown S, Jadhav A P, Haussen D C, Martins S O, Rebello L C, Demchuk A M, Goyal M, Ribo M, et al. Assessment of Optimal Patient Selection for Endovascular Thrombectomy Beyond 6 Hours After Symptom Onset: A Pooled Analysis of the AURORA Database. JAMA Neurol. 2021; 78:1064-1071
11. Fonarow G C, Smith E E, Saver J L, Reeves M J, Hernandez A F, Peterson E D, Sacco R L, Schwamm L H. Improving door-to-needle times in acute ischemic stroke: the design and rationale for the American Heart Association/American Stroke Association's Target: Stroke initiative. Stroke. 2011; 42:2983-2989
12. Henninger N, Fisher M. Extending the Time Window for Endovascular and Pharmacological Reperfusion. Transl Stroke Res. 2016; 7:284-293
13. Goyal M, Menon B K, van Zwam W H, Dippel D W, Mitchell P J, Demchuk A M, Davalos A, Majoie C B, van der Lugt A, de Miquel M A, et al. Endovascular thrombectomy after large-vessel ischaemic stroke: a meta-analysis of individual patient data from five randomised trials. Lancet. 2016; 387:1723-1731
14. Albers G W, Marks M P, Kemp S, Christensen S, Tsai J P, Ortega-Gutierrez S, McTaggart R A, Torbey M T, Kim-Tenser M, Leslie-Mazwi T, et al. Thrombectomy for Stroke at 6 to 16 Hours with Selection by Perfusion Imaging. N Engl J Med. 2018; 378:708-718
15. Tovmasyan A, Sheng H, Weitner T, Arulpragasam A, Lu M, Warner D S, Vujaskovic Z, Spasojevic I, Batinic-Haberle I. Design, mechanism of action, bioavailability and therapeutic effects of mn porphyrin-based redox modulators. Med Princ Pract. 2013; 22:103-130
16. Batinic-Haberle I, Tovmasyan A, Spasojevic I. Mn Porphyrin-Based Redox-Active Drugs: Differential Effects as Cancer Therapeutics and Protectors of Normal Tissue Against Oxidative Injury. Antioxid Redox Signal. 2018; 29:1691-1724
17. Barbosa A M, Sarmento-Neto J F, Menezes Filho J E R, Jesus I C G, Souza D S, Vasconcelos V M N, Gomes F D L, Lara A, Araujo J S S, Mattos S S, et al. Redox-Active Drug, MnTE-2-PyP (5+), Prevents and Treats Cardiac Arrhythmias Preserving Heart Contractile Function. Oxid Med Cell Longev. 2020; 2020:4850697
18. Tovmasyan A, Bueno-Janice J C, Jaramillo M C, Sampaio R S, Reboucas J S, Kyui N, Benov L, Deng B, Huang T T, Tome M E, et al. Radiation-Mediated Tumor Growth Inhibition Is Significantly Enhanced with Redox-Active Compounds That Cycle with Ascorbate. Antioxid Redox Signal. 2018; 29:1196-1214
19. Batinic-Haberle I, Tovmasyan A, Huang Z, Duan W, Du L, Siamakpour-Reihani S, Cao Z, Sheng H, Spasojevic I, Alvarez Secord A. H202-Driven Anticancer Activity of Mn Porphyrins and the Underlying Molecular Pathways. Oxid Med Cell Longev. 2021; 2021:6653790
20. Batinic-Haberle I, Tome M E. Thiol regulation by Mn porphyrins, commonly known as SOD mimics. Redox Biol. 2019; 25:101139
21. Mackensen G B, Patel M, Sheng H, Calvi C L, Batinic-Haberle I, Day B J, Liang L P, Fridovich I, Crapo J D, Pearlstein R D, et al. Neuroprotection from delayed postischemic administration of a metalloporphyrin catalytic antioxidant. J Neurosci. 2001; 21:4582-4592
22. Sheng H, Enghild J J, Bowler R, Patel M, Batinic-Haberle I, Calvi C L, Day B J, Pearlstein R D, Crapo J D, Warner D S. Effects of metalloporphyrin catalytic antioxidants in experimental brain ischemia. Free Radic Biol Med. 2002; 33:947-961
23. Sheng H, Yang W, Fukuda S, Tse H M, Paschen W, Johnson K, Batinic-Haberle I, Crapo J D, Pearlstein R D, Piganelli J, et al. Long-term neuroprotection from a potent redox-modulating metalloporphyrin in the rat. Free Radic Biol Med. 2009; 47:917-923
24. Sheng H, Spasojevic I, Tse H M, Jung J Y, Hong J, Zhang Z, Piganelli J D, Batinic-Haberle I, Warner D S. Neuroprotective efficacy from a lipophilic redox-modulating Mn (III)N-Hexylpyridylporphyrin, MnTnHex-2-PyP: rodent models of ischemic stroke and subarachnoid hemorrhage. J Pharmacol Exp Ther. 2011; 338:906-916
25. Bowler R P, Sheng H, Enghild J J, Pearlstein R D, Warner D S, Crapo J D. A catalytic antioxidant (AEOL 10150) attenuates expression of inflammatory genes in stroke. Free Radic Biol Med. 2002; 33:1141-1152
26. Rajic Z, Tovmasyan A, Spasojevic I, Sheng H, Lu M, Li A M, Gralla E B, Warner D S, Benov L, Batinic-Haberle I. A new SOD mimic, Mn (III) ortho N-butoxyethylpyridylporphyrin, combines superb potency and lipophilicity with low toxicity. Free Radic Biol Med. 2012; 52:1828-1834
27. Yokoo N, Sheng H, Mixco J, Homi H M, Pearlstein R D, Warner D S. Intraischemic nitrous oxide alters neither neurologic nor histologic outcome: a comparison with dizocilpine. Anesth Analg. 2004; 99:896-903
28. Taninishi H, Pearlstein M, Sheng H, Izutsu M, Chaparro R E, Goldstein L B, Warner D S. Video training and certification program improves reliability of postischemic neurologic deficit measurement in the rat. J Cereb Blood Flow Metab. 2016; 36:2203-2210
29. Badhiwala J H, Nassiri F, Alhazzani W, Selim M H, Farrokhyar F, Spears J, Kulkarni A V, Singh S, Alqahtani A, Rochwerg B, et al. Endovascular Thrombectomy for Acute Ischemic Stroke: A Meta-analysis. JAMA. 2015; 314:1832-1843
30. Berkhemer O A, Majoie C B, Dippel D W, Investigators M C. Endovascular therapy for ischemic stroke. N Engl J Med. 2015; 372:2363
31. Broderick J P, Berkhemer O A, Palesch Y Y, Dippel D W, Foster L D, Roos Y B, van der Lugt A, Tomsick T A, Majoie C B, van Zwam W H, et al. Endovascular Therapy Is Effective and Safe for Patients With Severe Ischemic Stroke: Pooled Analysis of Interventional Management of Stroke III and Multicenter Randomized Clinical Trial of Endovascular Therapy for Acute Ischemic Stroke in the Netherlands Data. Stroke. 2015; 46:3416-3422
32. Saver J L, Goyal M, Bonafe A, Diener H C, Levy E I, Pereira V M, Albers G W, Cognard C, Cohen D J, Hacke W, et al. Stent-retriever thrombectomy after intravenous t-PA vs. t-PA alone in stroke. N Engl J Med. 2015; 372:2285-2295
33. Berkhemer O A, Fransen P S, Beumer D, van den Berg L A, Lingsma H F, Yoo A J, Schonewille W J, Vos J A, Nederkoorn P J, Wermer M J, et al. A randomized trial of intraarterial treatment for acute ischemic stroke. N Engl J Med. 2015; 372:11-20
34. Kaesmacher J, Bellwald S, Dobrocky T, Meinel T R, Piechowiak E I, Goeldlin M, Kurmann C C, Heldner M R, Jung S, Mordasini P, et al. Safety and Efficacy of Intra-arterial Urokinase After Failed, Unsuccessful, or Incomplete Mechanical Thrombectomy in Anterior Circulation Large-Vessel Occlusion Stroke. JAMA Neurol. 2020; 77:318-326
35. Castonguay A C, Jumaa M A, Zaidat O O, Haussen D C, Jadhav A, Salahuddin H, Zaidi S F. Insights Into Intra-arterial Thrombolysis in the Modern Era of Mechanical Thrombectomy. Front Neurol. 2019; 10:1195
36. Heiferman D M, Li D D, Pecoraro N C, Smolenski A M, Tsimpas A, Ashley W W, Jr. Intra-Arterial Alteplase Thrombolysis during Mechanical Thrombectomy for Acute Ischemic Stroke. J Stroke Cerebrovasc Dis. 2017; 26:3004-3008
37. Rajic Z, Tovmasyan A, de Santana O L, Peixoto I N, Spasojevic I, do Monte S A, Ventura E, Reboucas J S, Batinic-Haberle I. Challenges encountered during development of Mn porphyrin-based, potent redox-active drug and superoxide dismutase mimic, MnTnBuOE-2-PyP (5+), and its alkoxyalkyl analogues. J Inorg Biochem. 2017; 169:50-60
38. Gad S C, Sullivan D W, Jr., Spasojevic I, Mujer C V, Spainhour C B, Crapo J D. Nonclinical Safety and Toxicokinetics of MnTnBuOE-2-PyP5+ (BMX-001). Int J Toxicol. 2016; 35:438-453

The foregoing is illustrative of the present invention, and is not to be construed as limiting thereof. The invention is defined by the following claims, with equivalents of the claims to be included therein.

1. A method of reducing an ischemia-reperfusion injury in a subject in need thereof, the method comprising:
intra-arterially administering a redox active compound that comprises a metal to the subject, thereby reducing the ischemia-reperfusion injury in the subject.

2. The method of claim 1, wherein the redox active compound is a meso-substituted metalloporphyrin.

3. The method of claim 1, wherein the redox active compound has a structure represented by Formula I: 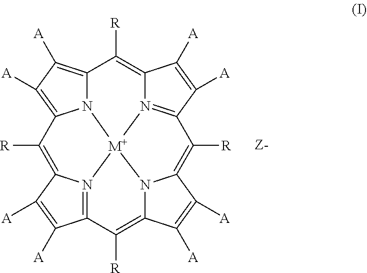
wherein:
each R is independently substituted or unsubstituted aryl, heteroaryl, cycloalkyl, or heterocycloalkyl;
each A is an independently selected hydrogen, or an electron-withdrawing or electron donating group;
M is a metal; and
Z⁻ is a counterion;
or a pharmaceutically acceptable salt thereof.

4. The method of claim 1, wherein the redox active compound has a structure of:
Formula A1 or A2: 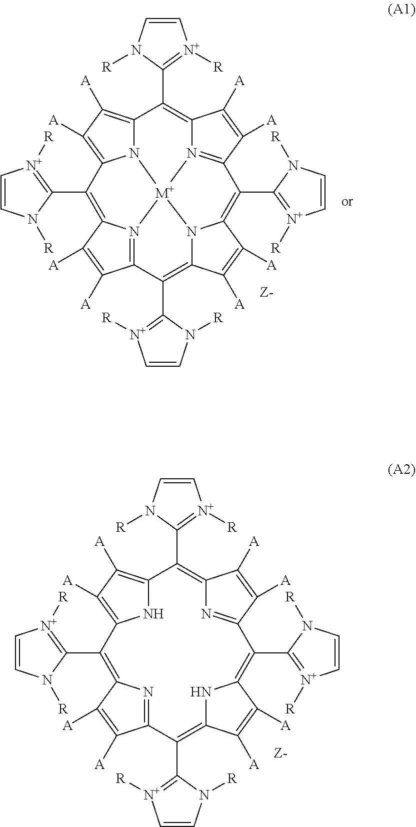
wherein:
each R is independently a C1-12 alkyl,
each A is independently a hydrogen or an electron withdrawing group,
M is a metal selected from the group consisting of manganese, iron, copper, cobalt, nickel and zinc, and
Z⁻ is a counterion,
or a pharmaceutically acceptable salt thereof.

5. The method of claim 1, wherein the redox active compound has a structure of Formula B1: 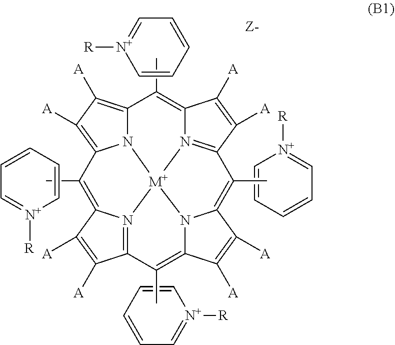
wherein:
each R is independently a C1-12 alkyl or C1-C12 alkoxyalkyl;
each A is independently a hydrogen or an electron withdrawing group;
M is metal selected from the group consisting of manganese, iron, copper, cobalt, and nickel; and
Z⁻ is a counterion;
or a pharmaceutically acceptable salt thereof.

6. The method of claim 1, wherein the redox active compound is MnTE-2-PyP⁵⁺ and has the structure: 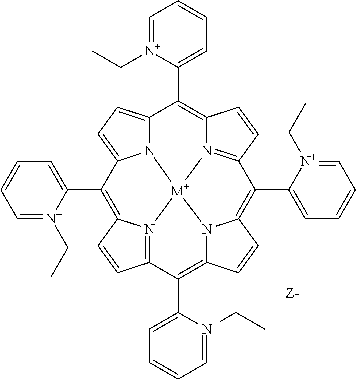
wherein Z⁻ is a counterion and M⁺ is manganese;
or a pharmaceutically acceptable salt thereof.

7. The method of claim 1, wherein said redox active compound has a structure of Formula C1: 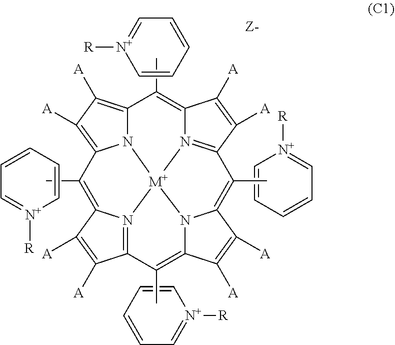
wherein:
each R is independently hydrogen or —(CH₂)ₘCH₂OX;
m is 1 or 2;
X is C1-12 alkyl;
each A is independently a hydrogen or an electron withdrawing group;
M is metal selected from the group consisting of manganese, iron, copper, cobalt, and nickel, and
Z⁻ is a counterion;
or a pharmaceutically acceptable salt thereof.

8. The method of claim 7, wherein said redox active compound has the structure: 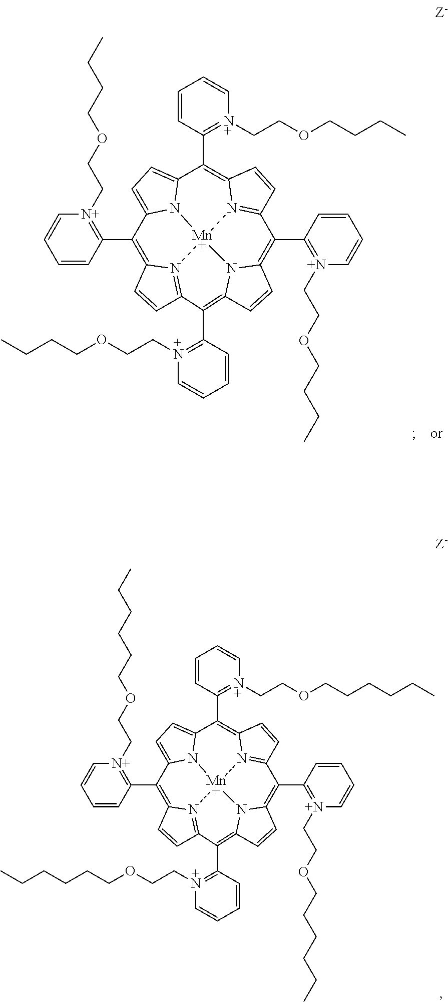
wherein Z⁻ is a counterion;
or a pharmaceutically acceptable salt thereof.

9. The method of claim 7, wherein said redox active compound is BMX-001 and has the structure: 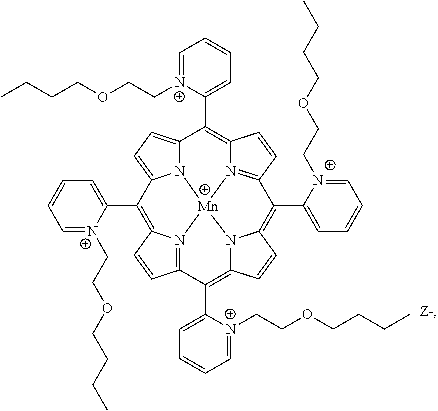
wherein Z⁻ is a counterion;
or a pharmaceutically acceptable salt thereof.

10. The method of claim 1, wherein the redox active compound is micronized and/or wherein the redox active compound has a D90 particle size of less than 50 microns.

11. (canceled)

12. The method of claim 1, wherein, responsive to the intra-arterially administering, the redox active compound is delivered to ischemic tissue in the subject.

13. The method of claim 1, wherein the intra-arterially administering comprises intra-arterially administering the redox active compound into the carotid artery of the subject and/or the middle cerebral artery of the subject.

14. The method of claim 1, wherein the intra-arterially administering comprises intra-arterially administering the redox active compound using an intra-arterial catheter.

15-16. (canceled)

17. The method of claim 1, wherein the intra-arterially administering comprises intra-arterially administering the redox active compound prior to and/or after removal of a catheter and/or filament and/or after reperfusion.

18. The method of claim 1, wherein the intra-arterially administering comprises intra-arterially administering the redox active compound directly to a clot in the subject.

19. The method of claim 1, wherein the intra-arterially administering comprises intra-arterially administering the redox active compound to the subject within about 12 hours of the ischemia-reperfusion injury.

20. The method of claim 1, wherein the intra-arterially administering comprises intra-arterially administering the redox active compound to the subject in an amount of about 3 μg/kg to about 500 μg/kg.

21-26. (canceled)

27. The method of claim 1, wherein the intra-arterially administering is performed prior to, during, and/or after a thrombectomy, embolectomy, angioplasty, and/or a cardiac catheterization performed on the subject.

28-29. (canceled)

30. The method of claim 1, wherein the method improves the ischemia-reperfusion injury outcome in the subject.

31-40. (canceled)

41. A method of increasing viability of an organ awaiting transplant and/or reducing transplant failure in a subject, the method comprising contacting the organ and a redox active compound that comprises a metal, thereby increasing the viability of the organ awaiting transplant and/or reducing transplant failure in the subject.

42-56. (canceled)
